(19) United States
(12) Patent Application Publication
(10) Pub. No.: US 2013/0123309 A1
(43) Pub. Date: May 16, 2013

(54) METHOD FOR REDUCING MICROBIAL LOAD FOR INDIVIDUALS WITH ELEVATED RISK CONDITIONS

(71) Applicant: OASIS CONSUMER HEALTHCARE LLC, Cleveland, OH (US)

(72) Inventors: Afif Mahmoud Ghannoum, Shaker Heights, OH (US); Brian Vincent Sokol, Vermilion, OH (US)

(73) Assignee: OASIS CONSUMER HEALTHCARE LLC, Cleveland, OH (US)

(21) Appl. No.: 13/734,470

(22) Filed: Jan. 4, 2013

Related U.S. Application Data
(63) Continuation-in-part of application No. 13/448,926, filed on Apr. 17, 2012.
Application No. 13/448,926 is a continuation-in-part of application No. 13/448,957, filed on Apr. 17, 2012.
Application No. 13/448,957 is a continuation-in-part of application No. PCT/US12/33921, filed on Apr. 17, 2012.
(60) Provisional application No. 61/477,147, filed on Apr. 19, 2011.
(60) Provisional application No. 61/477,147, filed on Apr. 19, 2011.
(60) Provisional application No. 61/477,147, filed on Apr. 19, 2011.
(60) Provisional application No. 61/725,375, filed on Nov. 12, 2012.

(51) Int. Cl. A61K 31/4425

(52) U.S. Cl. CPC A61K 31/4425
USPC 514/358

(57) ABSTRACT

A method for reducing risk of infection from microorganisms for certain elevated-risk individuals, includes: administering a barrier-forming composition in a therapeutically effective amount to a surface, the surface comprising a mucosa, or a mucosal or skin lesion of an individual. The individual has an elevated risk condition selected from: an elevated risk of being exposed to harmful viral, bacterial, or fungal microorganisms; a condition causing an elevated risk of infection from viral, bacterial, or fungal microorganisms; and an elevated risk of serious complications resulting from an infection caused by viral, bacterial, or fungal microorganisms. The method includes forming a barrier coating on the surface that is active to trap, and kill or neutralize microorganisms encountered by the barrier coating and effectively reducing the microbial load on the surface and continuing to administer the barrier-forming composition in a therapeutically effective amount to the surface, for one time or more in a 24 hour period.

### CROSS-REFERENCE TO RELATED APPLICATIONS

This application is a continuation-in-part of U.S. application Ser. No. 13/448,926, filed on Apr. 17, 2012, entitled, “Method of Inhibiting Harmful Microorganisms and Barrier-Forming Composition Therefor;” a continuation-in-part of U.S. application Ser. No. 13/448,957, filed on Apr. 17, 2012, entitled, “Method of Inhibiting Harmful Microorganisms and Barrier-Forming Composition Therefor;” and a continuation-in-part of PCT/US12/33921, filed on Apr. 17, 2012, and entitled “Method of Inhibiting Harmful Microorganisms and Barrier-Forming Composition Therefor.” All three of which, in turn, claim the benefit of priority to U.S. provisional application No. 61/477,147, filed on Apr. 19, 2011, entitled “Compositions, Methods of Use, and Methods of Making Barrier Products.” This application also claims the benefit of priority to U.S. provisional application No. 61/725,375, filed on Nov. 12, 2012. All of these prior applications are incorporated herein by reference for all purposes.

### FIELD

This disclosure relates to barrier-forming compositions and methods for treating mucosal surfaces to prevent the spread of communicable diseases.

### BACKGROUND

There has been a longstanding need for devices, compositions, and other treatments that will effectively prevent communicable diseases, especially for individuals with elevated risks to infection, such as individuals who are immunocompromised. Attempts at solving this problem include wearing masks or respirators and avoiding or quarantining of individuals or animals that are known or expected to be sick or carrying germs. Such approaches are common in hospital settings, and such individuals often wear masks while encountering contaminated environments such as public transportation or public gathering places.

While numerous solutions exist for killing microorganisms once they have contacted a person or animal, the effectiveness of such solutions is dependent on quick recognition of the germ contact and application of the germ-killing composition prior to the microorganism binding to a host cell on mucosal surface or a disrupted area of the skin, whereby it would enter the body and infect the individual or otherwise infect the individual. For example, washing with an anti-bacterial soap may be effective for killing germs on the hands; however, it is very easy for a person to unwittingly touch a contaminated surface and put their hands near or in their mouth or nose before washing their hands. In addition, many current solutions only kill one group of harmful microorganisms (bacteria, viruses, and fungi) leaving other groups unaffected.

Physical devices such as masks are uncomfortable and unsightly and herbal remedies have unproven results, and solutions for killing germs that have already contacted the body are often ineffective for prevention of infection since they are intermittent, transitory options that do not provide sustained protection, and do not always offer broad spectrum protection against, bacteria, viruses, and fungi.

### SUMMARY

In an embodiment, a method for reducing risk of infection from microorganisms for certain elevated-risk individuals, includes: administering a barrier-forming composition in a therapeutically effective amount to a surface, the surface comprising a mucosa, or a mucosal or skin lesion of an individual. The individual has an elevated risk condition selected from: an elevated risk of being exposed to harmful viral, bacterial, or fungal microorganisms; a condition causing an elevated risk of infection from viral, bacterial, or fungal microorganisms; and an elevated risk of serious complications resulting from an infection caused by viral, bacterial, or fungal microorganisms. The method includes forming a barrier coating on the surface that is active to trap, and kill or neutralize microorganisms encountered by the barrier coating and effectively reducing the microbial load on the surface and continuing to administer the barrier-forming composition in a therapeutically effective amount to the surface, for one time or more in a 24 hour period.

In an embodiment, a method for reducing the microbial load in immunocompromised individuals includes: administering a barrier-forming composition that comprises an antimicrobial to a surface, the surface comprising an oral, nasal, or pharyngeal mucosa or a skin lesion of an immunocompromised individual; forming a barrier coating on the surface that is active to trap, and kill or neutralize microorganisms encountered by the barrier coating for a duration of at least about one hour, thereby effectively reducing the microbial load of the surface. The barrier-forming composition meets the following requirements:

about 0.0001%≦C≦about 0.4%;

about 0.07%≦H≦about 70%; and

0.0005%<A or

about 0%≦C≦about 0.4%;

about 55%≦H≦about 70%; and

0.0005%<A

wherein all percentages are by weight of the total composition;

wherein C is the carbohydrate gum; H is the humectant; and A is the antimicrobial agent.

The articles “a” and “the,” as used herein, mean “one or more” unless the context clearly indicates to the contrary.

The terms “item” and “apparatus” are used synonymously herein.

The term “therapeutic,” as used herein, is meant to also apply to preventative treatment.

The term “or,” as used herein, is not an exclusive or, unless the context clearly indicates to the contrary.

The use of the term “individual” or “mammal” herein, means a human or animal commonly defined as a mammal.

The term “lesion” is used herein interchangeably with the term “disruption.”

The terms “block” or “blocking” as used herein, include blocking passage by trapping.

### BRIEF DESCRIPTION OF THE DRAWINGS

FIG. 1 is a depiction of a proposed mechanism of antimicrobial activity in an embodiment of the barrier-forming composition.

FIG. 2 is a schematic showing the formation of a barrier on a mucosal surface, as described in Example 2.

FIG. 3 is a graph showing the repair process as a percentage on a wounded epithelial cell sample through cell growth and migration after 6 hours to cover the scratched space in each of Examples 11-15.

FIG. 4 shows microscopic photographs showing epithelial cell growth and migration on both untreated control Example 15 and treated Examples 16 and 17 on a wounded epithelial sample.

FIG. 5 shows photos of magnified cross-sections of the barrier-forming composition-treated and untreated engineered human oral mucosa (EHOM) of Examples 11-15.

FIG. 6 is graph showing an LDH assay of Examples 16-19 and 20-25.

FIG. 7 is a schema showing the method of evaluation of microbial growth in the upper and lower chambers of an EHOM assay, as described in Examples 27-28.

FIG. 8 show photographs of agar media plates showing microbial growth in the upper and lower chambers of an EHOM assay, as described in Examples 27-28.

FIG. 9 shows photographs of magnified cross-sections of the barrier-forming composition-treated and untreated engineered human oral mucosa (EHOM) of Examples 31-32.

FIG. 10 shows photographs of microbial growth on untreated EHOM or EHOM treated with an example barrier-forming composition, followed by infection with C. albicans, as described in Examples 33-40.

FIG. 11 shows photographs of microbial growth on untreated EHOM or EHOM treated with formulations followed by infection with S. mutans, as described in Examples 33-40.

FIG. 12 shows photographs of microbial growth from “flow-through” media (collected from the lower chamber) of EHOM treated with an example barrier-forming composition, as described in Example 33-40.

FIG. 13 presents graphs showing LDH release by EHOM treated with saline (control) or example barrier-forming compositions, followed by infection with (A) C. albicans or (B) S. mutans, as described in Examples 40-47.

FIG. 14 is a graph showing post-antimicrobial effect of barrier-forming compositions against bacteria and fungi, as described in Examples 48-61 and 61-69.

FIG. 15 shows scanning electron micrographs of S. sanguis, C. albicans, and S. mutans, untreated or treated with barrier-forming composition, as described in Examples 71-76.

FIG. 16 presents graphs depicting activity of an example barrier-forming composition against biofilms formed by bacteria and fungi, as described in Examples 77-79.

FIG. 17 is a graph showing activity of an example barrier-forming composition on microbial biofilms after a 1-min exposure, as described in Examples 80-81.

FIG. 18 presents fluorescent microscopy photographs showing the effect of an example barrier-forming composition on cytopathic effects (CPE) of influenza (H1N1)-infected MDCK cells, as described in Examples 85-86.

FIG. 19 presents fluorescent microscopy photographs showing the effect of an example barrier-forming composition on against H1N1 virus, as described in Examples 85-86.

FIG. 20 is a graph showing levels of influenza virus in infected barrier-forming composition treated and -untreated cells, as determined by quantitative PCR, as described in Examples 87-88.

FIG. 21 is a graph showing direct antiviral activity of example barrier-forming compositions prepared with or without preservatives and antimicrobial agent (CPC) against influenza virus, determined using quantitative PCR, as described in Examples 89-91.

FIG. 22 shows the activity of an example barrier-forming composition against H1N1 virus over a 6 hour time period. Panel (A) is a graph showing a percent inhibition in viral growth compared to an untreated control. Panels (B) and (C) are micrographs of (B) untreated and (C) barrier-forming composition treated cells.

FIG. 23 is a graph showing the activity of formulations against HIV, as described in Examples 94-96.

FIG. 24 is a Western blot showing activity of Example 8 against Epstein-Barr Virus (EBV), as described in Example 97.

FIG. 25 is a graph showing LDH levels as an indicator of cellular integrity in untreated (control) EHOM or EHOMs tissues exposed to Examples 5-7, as described in Examples 154-159.

FIG. 26 shows representative photographs of a wounded oral epithelial cell culture treated with Example 3 (5% dilution) for 10 minutes, immediately after the wound (panel A), after about 6 hours (panel D), and after about 24 hours (panel E), as described in Example 160. Panels B and C show an equivalent wound on an untreated control confluent culture of oral epithelial cells after about 6 hours and about 24 hours, respectively.

FIG. 27 are photographs demonstrating the ability of an example barrier-forming composition to coat the oral mucosal surface.

FIG. 28 are photographs showing time-lapse microscopy of bacterial growth after a 1 minute exposure to an example barrier-forming composition, as described in Examples 162-163. Images represent bacterial growth after 20 min, 120 min, or 360 min post-exposure.

FIG. 29 is a graph showing the effect of a single dose of an example barrier-forming composition on oral microbial burden of a healthy individual, as described in Example 164-166. (A)—Microbial load in CFUs, (B) reduction in microbial load (%) compared to baseline.

FIG. 30 is a graph showing the effect of an example barrier-forming composition on levels of oral microbes over a 5-day period in three healthy adults, as described in Examples 167-169.

FIG. 31 is a graph showing the effect of an example barrier-forming composition on microbial burden of the oral cavity after 5-day usage in 31 healthy subjects, as described in Examples 170-198.

FIG. 32 is a graph showing the microbial load in oral samples obtained from three representative study participants, as described in Examples 170-198.

FIG. 33 shows is a schema describing the in vitro filter insert-based model to evaluate penetration of microbes across the barrier formed by example barrier-forming compositions, as described in Examples 199-205.

FIG. 34 is a set of photographs showing growth of MRSA biofilms on the surface of silicone elastomer discs treated with PBS (control, A, C, E) and Example 7 barrier forming composition (B, D, F), as described in Examples 219-224.

FIG. 35 is a set of photographs showing cell monolayers treated with an embodiment of the barrier-forming composition, Example 252, for varying time periods (a), (b), and (c), and a control Example 253 (d).

FIG. 36 is a set of immunofluorescence photographs showing cell monolayers treated with an embodiment of the barrier-forming composition, Example 252, for varying time periods (a), (b), and (c), and a control Example 253 (d).

### DETAILED DESCRIPTION

This application discloses a stable barrier-forming composition for reducing microbial load in individuals (mammals). In an embodiment, the barrier-forming composition is non-toxic to mammals and safe in a therapeutically effective amount. The barrier-forming composition forms a barrier coating that inhibits the passage of active pathogenic microbes through to the other side. The barrier-forming composition includes an antimicrobial component to inhibit microbial growth through static or cidal activity for an extended period of time. The combined barrier coating and antimicrobial synergistically act to block, neutralize, and/or kill microorganisms recently deposited on the treated surface and/or microorganisms that subsequently come into contact with an exposed (top) surface of the barrier coating, thereby providing a long-lasting antimicrobial that is significantly more powerful than just an antimicrobial alone. The barrier coating is effective to trap and kill or neutralize microorganisms already present on the treated surface and/or to trap and kill or neutralize microorganisms that are subsequently deposited on top of the barrier, i.e., the exposed surface of the barrier coating, after the administering of the barrier forming composition is performed. Without being bound by theory, the mechanism of action of the barrier-forming composition disclosed herein is based on a synergistic dual-action mechanism, in which germs are trapped in the formed barrier coating, and subsequently killed by the antimicrobial active ingredient. In an embodiment, the barrier-forming composition is not hydrophilic, which, without being bound by theory, is theorized to enhance its sustained effectiveness.

As shown in the Examples below, the properties of the barrier-forming composition and its effectiveness to prevent a wide variety of communicable diseases and reduce microbial load were assessed using at least ten different approaches based on: (1) an in vitro anti-microbial susceptibility testing; (2) an in vitro time kill assay; (3) an in vitro biofilm model; (4) an in vitro filter insert-based model, (5) an in vivo-like engineered human oral mucosa (EHOM) model; (6) electron microscopy evaluation; (7) hydrophobicity assay; (8) physico-chemical compatibility assays; (9) cell culture-based model using monolayer of human cell lines; and (10) human clinical trials.

The barrier-forming composition is particularly useful for individuals that have an elevated risk condition. For example, an elevated risk of being exposed to harmful viral, bacterial, or fungal microorganisms; a condition causing an elevated risk of infection from viral, bacterial, or fungal microorganisms; or an elevated risk of serious complications resulting from an infection caused by viral, bacterial, or fungal microorganisms. For example, the method and composition described herein may be particularly useful when a human, or more generally, a mammal, has a disrupted skin or mucosa or has a condition resulting in an immunocompromised state or is otherwise at a greater risk for infection. Such persons may benefit from administration of the barrier-forming composition in repeated doses for ongoing proactive protection and reduction in overall microbial load. In an embodiment, the administration may be in response to identification of a contaminated environment or observation of a contamination event.

Various conditions that cause an elevated risk of infection from viral, bacterial, or fungal microorganisms include, but are not limited to: having an immune system that has been impaired by disease or medical treatment, being the recipient of a transplant, such as an organ transplant, a skin graft, or a bone marrow transplant, having undergone surgery within 30 days, undergoing ventilator treatments, having HIV, AIDS, cancer, COPD, or diabetes, having lesions on the skin or mucosa, or having a condition that causes lesions on the skin or mucosa.

In the case of a skin or mucosal disruption or lesion, such may be caused by a wound, scratch, or other opening in the skin or mucosa. The skin and the mucosa of the oral cavity and gastrointestinal (GI) tract serve as an important mechanical barrier that helps to prevent a local or systemic invasion of various microbes and the absorption of microbial products that are normally present in the oral cavity and the lumen of the gut. “Gastrointestinal mucosal injury in experimental models of shock, trauma, and sepsis,” Crit. Care Med. 1991; 19:627-41.). Derangement in the barrier function of the mucosa plays a central role in the pathophysiology of systemic infection. In other words, disruption of the skin or mucosa will lead or increase the risk of infections.

Elimination or reduction of the risk of a breach in the first line of defense is important, and the maintenance of mucosal or skin integrity is important. (Anders Heimdahl, “Prevention and Management of Oral Infections in Cancer Patients” Supportive Care in Cancer, Vol. 7, No. 4, 224-228 (1999).) Thus, having intact skin or mucosa is an important host defense against systemic infection, particularly in immunocompromised patients (e.g. cancer patients). (Shahab A. Khan, John R. Wingard, “Infection and Mucosal Injury,” Cancer Treatment Journal of the National Cancer Institute, Monographs No. 29 (2001). A barrier-forming composition that blocks and kills harmful microorganisms and that does not interfere with healing of a disrupted skin or mucosa is a unique and unexpected solution to the susceptibility of the problems of those with disrupted skin or mucosa, particularly those that also have immunodeficiency.

In addition, certain medical conditions are known to cause an increased incidence of skin or mucosal lesions. For example, sexually transmitted diseases, including but not limited to HIV and AIDS, cancer, psoriasis, acne, diabetes, and lupus.

The elevated risk condition may be due to an elevated risk of serious complications resulting from an infection caused by viral, bacterial, or fungal microorganisms. In this case, the elevated risk is not necessarily because infection is more likely, but that if infection does occur it is likely to have more serious consequences due to complications. The elevated risk may, for example, be due to, but not limited to, one or more of the following conditions: being immunocompromised in general, undergoing chronic steroid treatment, being under 19 and undergoing long-term aspirin treatment, being morbidly obese, being pregnant, being 65 years of age or older, children younger than two years of age, HIV, AIDS, cancer, metabolic disorder, mitochondrial disorder, liver disorder, kidney disorder, asthma, blood disorders, endocrine disorders, heart disease, chronic lung disease, cerebral palsy, epilepsy, stroke, muscular dystrophy, and spinal cord injury.

In another elevated risk condition, the individual has an elevated risk of exposure to harmful microorganisms for prolonged periods of time, such as every day, or at least four or five days a week. In this elevated risk category, the individual does not necessarily have any elevated risk to getting infected once exposed to harmful microorganism or to having complications once infected, but instead has a higher risk of exposure to harmful microorganisms and/or a higher microbial load. In an example the risk of exposure is elevated due to the individual living or working on an airplane, train, bus, ship, boat, in a school, a library, a dormitory, a hotel, an apartment building, a courthouse, a correctional facility, an airport, a restaurant, a movie theater, a theater, a mall, a retail establishment, a special event arena, a special event stadium, a religious gathering place, a nursing home, hospital, other health-care facility, an office, or day-care facility.

In an embodiment, the barrier-forming composition and method described herein may be useful, for example, for prevention and/or treatment of infections from items that may be contaminated in activity related treatments, such as, for example, ventilator use (which would include medical devices related to the ventilator and contacting the patient). In an embodiment the contaminated item is a medical apparatus, or a dental apparatus. Having exposure to of a medical or dental device such as a ventilator may be considered as both an elevated risk of exposure and an elevated risk of infection. A nosocomial infection wherein an individual is already in an immunocompromised state and also is present in a hospital or other health-care facility environment may also be considered as an elevated risk of exposure and either one or both of an elevated risk of infection and an elevated risk of serious complication.

In an embodiment of a method for reducing risk of infection from microorganisms for certain elevated-risk individuals, the barrier-forming composition is administered in a therapeutically effective amount to a surface, the surface comprising an oral, nasal, or pharyngeal mucosa or a skin lesion of an individual. The barrier-forming composition, once administered, forms a barrier coating on the surface that is active to trap, and kill or neutralize microorganisms encountered by the barrier coating for a duration of at least about one hour, thereby effectively reducing the microbial load on the surface.

In an embodiment, in a continued dosage method of prevention or treatment, the barrier-forming composition may be administered in a therapeutically effective amount in a series of doses, such as, for example, about every 1 to 12 hours, about every 2 to 8 hours, or about every 4 to 6 hours. In another embodiment, the therapeutically effective amount of the barrier-forming composition is administered every about two to about twelve hours to the surface, such as every about three to about eight hours, or every about four to about six hours. Administering “every about two to about twelve hours” means one therapeutically effective dose being administered and then a second dose being administered about two hours later up to about twelve hours later, and additional doses, if taken, being administered in subsequent about two hour to about twelve hour increments.

In an embodiment, the barrier forming composition is administered in a therapeutically effective amount three times or more in a 24 hour period for five 24 hour periods or more, such as, for example, four to twelve times, or six to ten times for six days to ten days, or seven to thirty days. In another embodiment, the method of prevention can be continued, for example, for a day or more, such as for about two days to about a week, or up to a year, or for a person who has an incurable elevated risk condition, every day for the rest of their life. In an embodiment, the three or more doses may be taken only during daylight hours or an individual's waking hours, such as, for example, 6 AM to 6 PM, or 9 PM to 5 PM. In an embodiment, an individual may follow such a dosage regimen to provide protection during the entirety of their hours at a workplace or another public gathering place. In an embodiment, the continued dosage method may be preferred when the subject is in prolonged contact with a contaminated environment or item.

The dosage regimen may be different for persons having different elevated risk conditions. For example, individuals with an elevated risk of infection, such as immunocompromised patients, may administer the barrier-forming composition proactively throughout the day, everyday, and especially when in contaminated environments or upon observing a contamination event. Individuals having an elevated exposure risk, or an otherwise short-term elevated risk condition, such as someone having surgery, may, for example, administer the barrier-forming composition before or during exposure to high germ risk (contaminated) environments, like hospitals.

As shown in the Examples below, such a dosage regimen has been shown to substantially reduce microbial load in human clinical trials. In vivo testing has shown that about 80% of humans following the continued dosage method show a decrease of about 50% or greater of microbial load in the oral cavity over five days of treatment.

The mucosa that is treated, may, for example, be a mucosal surface in the oral cavity, the nasal cavity, throat, or the pharyngeal cavity, such as, the nasopharynx (epipharynx), the oropharynx (mesopharynx), or the laryngopharynx (hypopharynx). Beneficial results may also be gained by treating mucosa in other orifices of a mammal, including, but not limited to the ear canal.

In an embodiment, the barrier-forming composition is administered to a skin or mucosal lesion in response to identification of the skin or mucosal lesion. Subsequent dosages may be applied in accordance with dosage intervals discussed above. In an embodiment, the dosing regimen is ended when the lesion has healed, i.e. when it is covered with new skin or mucosal tissue.

In an embodiment of the method, the step of administering the barrier-forming composition occurs in response to one of the following conditions: (a) identifying a contaminated environment that the individual with an elevated risk condition is present in or is going to be present in, wherein the contaminated environment is known or expected to be contaminated with harmful viral, fungal, or bacterial microorganisms; or (b) observing a contamination event in an environment wherein the individual with an elevated risk condition is present in the environment or is going to be present in the environment. In an embodiment, the step of administering performed in response to the condition, may be the first administration of the barrier forming composition to begin a multi-dose regimen that continues until the individual is no longer present in the contaminated environment or the environment wherein the contamination event occurred. In an embodiment, second or subsequent therapeutically effective doses may be administered in response to the occurrence of the condition. For example, a person may first administer the barrier-forming composition when a contamination event occurs, and then continues to administer the barrier-forming composition in the dosage intervals described above until the individual leaves the vicinity of the contamination event. In another example after a dosage regimen has begun, the individual may administer a second or subsequent dose when a new contamination event is observed, so long as the administration occurs no sooner than the minimum of the described dosing time interval. In some situations, the minimum dosing interval may not be followed, such as persons in advanced stages of cancer or AIDs, or an individual that is in critical or a life-threatening condition.

A contamination event includes events such as an individual sneezing, coughing, or vomiting, or more generally where bodily fluids or matter have been deposited. In an embodiment, the contamination event is in the vicinity of the individual to trigger the administering response. The vicinity of the individual may be defined as being in the same room, vehicle, or within about 10 yards of the individual.

In an embodiment, the barrier-forming composition is applied to a mucosa of an individual with an elevated risk condition, such as an immunocompromised individual. The mucosa, may be an oral, pharyngeal, or nasal mucosa. In an embodiment, the administering step is performed in response to encountering an environment that is considered to be contaminated or in response to an observed contamination event. The barrier-forming composition provides a barrier coating on the mucosa surface that traps and kills microorganisms, such as those that are encountered subsequent to the application of the barrier-forming composition, thereby preventing or inhibiting active microorganisms from passing to the mucosa or causing infection.

In another embodiment, a barrier-forming composition is administered in a method preventing an infectious disease in mammal with a disrupted mucosa, such as for example an immunocompromised mammal. The disrupted area in a mucosa of the mammal is identified and a therapeutically effective amount of a barrier-forming composition is administered to at least the disrupted area of the mucosa of the mammal. The barrier-forming composition provides a barrier on the disrupted area of the mucosa that effectively inhibits active microorganisms from disseminating to a disrupted area of the mucosa.

In an embodiment of the method of preventing an infectious disease, a step includes identifying a contaminated environment or item that a mammal is expected to encounter. The contaminated environment is an environment such as an indoor or outdoor space or a proximity to another mammal or human that is known or expected to be contaminated with harmful viral, fungal, or bacterial microorganisms. The determination of whether a given environment may be contaminated may be based on the time of year, published information on flourishing diseases in the community, or observing others that appear to be sick or spreading germs by sneezing, etc. The latter factor may also be described as observing a contamination event.

Predicting or identifying whether the contaminated environment or item will be encountered can be a decision based on whether the mammal plans expects, or is expected to enter the environment or encounter the item in the near future. This may include estimating a time when the contaminated environment or item will be encountered. The barrier-forming composition may then be administered about twenty-four hours or less prior to the estimated time of encounter with the contaminated environment or item, such as, for example, about sixteen hours or less, about twelve hours or less, about six hours or less, or about two hour or less. The barrier-forming composition sets up quickly and should be operable to prevent or inhibit harmful microorganisms from infecting mucosa, for example, within less than one minute of application, such as within 30 seconds. Thus, it could be applied during the encounter with the contaminated environment or item and have effectiveness.

In an embodiment, the barrier-forming composition and method of treatment and prevention described herein may be useful, for example, for prevention of infections in environments such as hospitals and infections common in such environments that are contaminated with infectious microorganisms. As mentioned above, the methods and compositions disclosed herein may be especially applicable for immunocompromised patients or persons that spend significant working hours in health-care facilities. In addition, the barrier-forming composition may be useful for prevention of infections by microorganisms that commonly infect wounds.

The contaminated environment may include, for example, a public transportation vehicle, a public gathering place, and a room or vehicle containing a mammal known or expected to be ill, or a close proximity to a mammal known or expected to be ill. More information on environments commonly recognized as contaminated environments, such as an airplane, a nursery, and a health center, is disclosed in Yang, et al., “Concentrations and Size Distributions of Airborne Influenza A Viruses Measured Indoors at a Health Centre, a Day-Care Centre, and on Aeroplanes,” J. R. Soc. Interface (Feb. 7, 2011), which is incorporated herein by reference.

More specifically, in an embodiment, the public transportation vehicle may be, for example, an airplane, a bus, or a taxi. A public gathering place may be, for example, a doctor's office, a hospital, a school, a nursery, a church, a hotel, or a restaurant. The close proximity to a mammal known or expected to be ill may be, for example, within a one foot radius, or in the same motor vehicle with the mammal. A publicly used airplane may be mentioned as a common and particularly noteworthy example of an environment that many would identify as being a contaminated environment. As such persons that work on airlines or travel on public airplanes very frequently, e.g. two or three times per week may be considered to have the elevated risk condition due to increased exposure to microrganisms.

In an embodiment, the barrier-forming composition traps and/or kills or neutralizes all harmful microorganisms contacting the barrier-forming composition. In another embodiment, the barrier substantially traps and/or kills or neutralizes enough harmful microorganisms that contact the barrier-forming composition to inhibit or even stop them from causing an infectious disease. In the latter case, if the harmful microorganism's penetration of the barrier is slowed and/or diluted it will enhance the body's own ability to prevent the microorganisms from causing disease or widespread infection.

In vitro testing demonstrates that embodiments of the barrier-forming composition prevent all active bacteria from reaching the other side of the barrier for long periods, including about two hours or more, about six hours or more, about sixteen hours or more, and about twenty-four hours or more. In vitro testing shows that in viruses exposed to embodiments of the barrier-forming composition, growth may be inhibited for about two or more days (such as influenza), up to about nine days, (such as HIV), after which the viral count is still below the MIC for extended periods, such as about two or three additional days Inhibitory activity against influenza virus was observed for up to 48 hours.

The barrier-forming composition exhibited the activity to reduce the microbial load of humans in clinical trials. For example, a surprisingly effective reduction in microbial load of more than about 50% to about 99% from about one to about six hours after the administering step was demonstrated. In embodiments, the microbial load may be reduced by more than aboutl0%, by more than about 25%, or by more than about 70% from about one to about six hours after the administering step. Furthermore, these ranges of reduction in microbial load are sustainable for long periods of time with the disclosed dosing regimen.

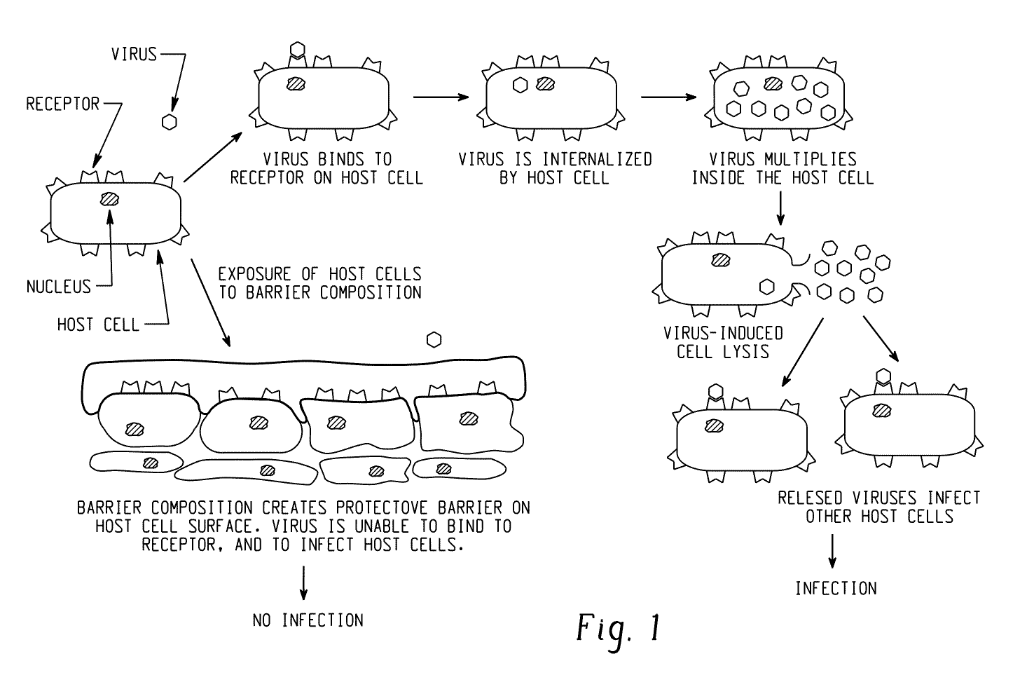

In an embodiment that illustrates a proposed mechanism of the barrier-forming composition in such a case, shown in FIG. 1, the barrier-forming composition provides anti-viral activity. When a virus comes into contact with a cell, it will bind to receptors on the host cell. Over time, 5 to 6 hours, or so, the virus is internalized by the host cell, the virus multiplies inside the host cell, and it induces cell lysis causing additional virus particles to infect other host cells.

In contrast, in a cell treated with the barrier-forming composition, a protective barrier is on the surface of the host cell. The barrier, which is thick enough to cover the cell and any receptors on the cell, prevents the virus particle from binding to the cell receptors. Thus, infection and lysis is also prevented. The barrier-forming composition retains the barrier for a long duration, such as a duration of about 1 hour of more, a duration of about 2 hours or more, a duration of about 6 hours or more, a duration of about 16 hours or more, a duration of about 16 hours to about 24 hours, or a duration of about 24 hours or more, thereby protecting host cells and preventing infection. The cidal or static antimicrobial activity is also retained for a long duration, such as about 2 hours or more, about 6 hours or more, about 16 hours or more, about 24 hours or more, or about 48 hours or more, thereby killing microorganisms and reducing microbial load These durations are applicable for viruses, bacteria, and fungi.

Harmful microorganisms are those known to cause infectious disease such as, for example, the treatment and prevention of infectious diseases, such as communicable diseases caused by microorganisms, such as Candida species (e.g. C. albicans, C. glabrata, C. krusei, C. tropicalis), Staphylococcus species (including methicillin-resistant S. aureus, MRSA), Streptococcus species (e.g. S. sanguis, S. oxalis, S. mitis, S. salivarius, S. gordonii, S. pneumoniae), Acinetobacter baumannii, Aggregatibacter actinomycetemcomitans, Fusobacterium nucleatum, and other microorganisms such as microorganisms that cause upper respiratory infections, and common cold (rhinovirus) and influenza viruses and Pneumonia, P. gingivalis, Y. enterocolitica, Acinetobacter bumanii, Aggregatibacter actinomycetemcomitans, Clostridium difficile, Bordetella pertussis, Burkholderia, Aspergillus fumigatus, Penicillium spp, Cladosporium, Klebsiella pneumoniae, Salmonella choleraesuis, Escherichia coli (0157:H7), Trichophyton mentagrophytes, Rhinovirus Type 39, Respiratory Syncytial Virus, Poliovirus Type 1, Rotavirus Wa, Influenza A Virus, Herpes Simplex Virus Types 1 & 2, and Hepatitis A Virus. In an embodiment, the barrier-forming composition and method of treatment and prevention described herein may be useful, for example, for prevention of sexually transmitted diseases such as, for example, infections caused by human immunodeficiency virus (HIV), Herpes simplex, or human papilloma virus (HPV).

The barrier-forming composition has shown effectiveness against microorganisms with a diameter of, for example, about 30 nm or greater, such as about 100 nm (HIV, spherical), about 100 to about 300 nm (influenza, spherical and elongated forms), about 120 nm to about 260 nm (EBV spherical/disk forms), and about 30 nm (rhinovirus, spherical). Thus, the barrier composition should also be effective against other microorganisms with diameters of about 30 nm, or greater than about 30 nm.

The barrier-forming composition has even shown powerful and surprising activity inhibiting biofilms, which can be very difficult to eradicate. In an embodiment, the method comprises administering the barrier-forming composition to a formed biofilm on a mucosa or lesion.

The microorganisms may be air-borne microorganisms. In an embodiment, the microorganisms are those that cause communicable diseases. In an embodiment, the microorganisms do not include those that cause allergic reactions or dental problems, such as, for example, cavities (caries), gingivitis, or seasonal allergies. Similarly, in an embodiment, the method of prevention does not solely or additionally prevent dental problems or allergic reactions, such as, for example, cavities (caries), gingivitis, or seasonal allergies.

In another embodiment, however, microorganisms, such as fungi that may generally be classified as allergens, other allergens, and airborne irritants to the mucosa, are also blocked by the barrier and the method.

In addition, the methods and compositions disclosed herein may be especially applicable for treating surfaces that immunocompromised persons will encounter.

In another embodiment, the method of treating a surface with the barrier forming composition includes identifying a contaminated surface, wherein the contaminated surface is known or expected to be contaminated with harmful viral, fungal, or bacterial microorganisms.

In an embodiment, the step of applying the barrier-forming composition occurs prior to or during a mammal that is not contaminated encountering the contaminated surface. In an embodiment, the application of the barrier-forming composition occurs in response to the identification of the surface as being contaminated or in response to an observation of a contamination event. For example, the barrier composition may be applied to a surface where a contamination event has occurred.

In another embodiment, the method of treating a surface with the barrier forming composition includes treating the surface with the barrier-forming composition proactively, regardless of whether the surface is known or expected to be contaminated with viral, fungal, or bacterial microorganisms. In an embodiment, the administered barrier coating traps and kills microorganisms that encounter the barrier coating after the treating step. As disclosed herein, the barrier coating is effective to kill microorganisms encountered for a long duration after the treatment step, thereby facilitating its effectiveness as a proactive treatment, which stands in contrast to prior art antimicrobial compositions that are not effective for proactive treatment, partly due to their ineffectiveness for long time-periods.

A therapeutically effective amount of the barrier composition includes an amount that is enough to coat the targeted mucosa or lesion with the barrier-forming composition to form a barrier coating that will result in a barrier layer forming on the mucosa or lesion. For example, about 100 microliters to about 10 ml, such as, for example, about 1 ml to about 8 ml, or about 2 ml to about 5 ml for a mouthwash formulation, or about 0.125 ml to about 2 ml, such as about 0.5 ml to about 1 ml for a spray formulation. The dosage amount may also be expressed in terms of a volume per square cm, such as, for example, from about 0.5 to about 50 μl/cm², such as, about 5 to about 40 μl/cm², or about 10 to about 25 μl/cm² for a mouthwash formulation; or for a spray formulation, for example, about 0.625 to about 10 μl/cm², such as, about 2.5 to about 5 μl/cm². Other delivery mediums, such as dissolvable strips, may have dosages derived from these ranges given the adjustments for concentrations and other factors known to those of skill in the art. In addition, the average thickness of the film formed on the mucosa from the barrier-forming composition may range, for example, from about 0.001 to about 0.2 mm, such as about 0.01 mm to about 0.1, or about 0.08 to about 0.15 mm. For example, for a given human or animal, the therapeutically effective amount can be determined based on the age or weight or size of the mammal to be treated, and the dosage may be those listed above. For non-human mammals, in particular, the dosage amount may be adjusted according to the per square cm values given above and the approximate surface area of the mucosal surface or body cavity to be treated.

Other delivery mediums, such as a liquid filled lozenge, may have dosages derived from these ranges given the adjustments for concentrations and other factors known to those of skill in the art.

The average thickness of the film or coating formed on the mucosal or mucosal or skin lesion surface from the barrier-forming composition may range, for example, from about 0.001 to about 0.2 mm, such as about 0.01 mm to about 0.1, or about 0.08 to about 0.15 mm.

A mechanical pump spray or an aerosolized spray device may be used. In the aerosolized embodiment, the barrier-forming composition may be mixed with common propellant agents, such as CO₂, nitrogen, and hydrocarbons. A bag-on-valve embodiment may also be used; however, the composition is stable enough so as not to require a separation of the propellant agent and the composition components.

An applicator, including but not limited to, a roll-on applicator, may be used with a dosage derived from the stated ranges given the adjustments for concentrations and other factors known to those of skill in the art.

A wipe, bandage or other applied material that is pretreated with the barrier technology may be used, which is then applied directly to the affected area, including disrupted mucosa. These may have dosages derived from the stated ranges given the adjustments for concentrations and other factors known to those of skill in the art.

In an embodiment, the barrier-forming composition comprises a carbohydrate gum (C), a humectant (H), and an antimicrobial agent (A), and the barrier-forming composition meets the following requirements:

about 0.0001%≦C≦about 0.4%;

about 0.07%≦H≦about 70%; and

0.0005%<A or

about 0%≦C≦about 0.4%;

about 55%≦H≦about 70%; and

0.0005%<A

All percentages are by weight of the total composition. The ranges in this embodiment reflect the demonstrated effectiveness of the germ killing power of the barrier-forming composition at very low dilutions against many microorganisms reported in MIC experiments in Table V below. After effective application, the barrier layer has antimicrobial cidal or static activity.

In another embodiment the barrier-forming composition meets the following requirements:

about 0.01%≦C≦about 0.4%;

about 4.5%≦H≦about 65%; and

0.0005%<A or

about 0%≦C≦about 0.4%;

about 55%≦H≦about 65%; and

0.0005%<A

All percentages are by weight of the total composition.

In another embodiment, the humectant of the barrier-forming composition meets the following requirements: about 0.07%≦H<1%. This low-humectant embodiment reduces the stickiness of the composition.

In an embodiment, the barrier-forming composition includes glycerin or one or more similar humectant substances. In an embodiment, the concentration of the humectant may range from about 0.07% to about 10% of the entire composition (by weight), such as about 3% to about 8%, 0.35% to less than 1%, or about 0.1% to less than 0.5%. In another embodiment, the humectant may range from about 2% to about 70% weight percent of the entire composition, such as, for example, about 4.5% to about 65%, about 7% to about 35%, or about 15% to about 45%. Humectants similar to glycerin may be classified generally as polyols. The humectants may be, for example, glycerin, sorbitol, xylitol, propylene glycol, polyethylene glycol, and mixtures thereof. In an embodiment, glycerin may be used at high concentrations such as about 55% to about 65% in the absence of a gum.

In an embodiment, the composition also includes a gum. The gum may be, for example, a polysaccharide, xanthan gum, gum Arabic, or guar gum. Such gums may be generally classified as carbohydrate gums that have an overall negative charge. In another embodiment, the gum may be, for example, xanthan gum, guar gum, gum Arabic, tragacanth, gum karaya, locust bean gum, carob gum, and pectin. These gums may also be generally classified as carbohydrate gums that have an overall negative charge. In an embodiment, the gum may be present in a weight percentage of the total composition ranging from about 0.0001% to about 0.4%, such as about 0.0005 to about 0.25%. In another embodiment, the gum may be present in a weight percentage of the total composition ranging from about 0.01% to about 0.4%, such as for example, about 0.25% to about 0.35%, about 0.05% to about 0.25%, or about 0.4%.

In an embodiment, the barrier composition comprises a humectant, an antimicrobial, and optionally a gum, wherein the gum, if present, is present in an amount of about 0.0001% to about 0.4% by weight of the total barrier-forming composition.

In an embodiment, an antimicrobial agent is present in the composition. For example, the composition may include one or more anti-viral agents, or antifungals or antibacterials or a combination thereof. In addition, the effect of such antimicrobials includes static and/or cidal activity.

The antimicrobial agent may include, but is not limited to cationic antimicrobial agents and pharmaceutically acceptable salts thereof, including, for example, monoquaternary ammonium compounds (QAC, cetrimide, benzalkonium chloride, cetalkonium chloride, cetylpyridinium chloride, myristalkonium chloride, Polycide), biquaternaries and bis-biguanides (Chlorhexidine, Barquat, hibitane), and biguanides, polymeric biguanides, polyhexamethylene biguanides, Vantocil, Cosmocil, diamidines, halogen-releasing agents including chlorine- and iodine-based compounds, silver and antimicrobial compounds of silver, peracetic acid (PAA), silver sulfadiazine, phenols, bisphenols, hydrogen peroxide, hexachloroprene, halophenols, including but not limited to chloroxylenol (4-chloro-3,5-dimethylphenol; p-chloro-m-xylenol).

In addition, the antimicrobial may also be or include: antibacterial agents, both cidal and static, and different classes, for example tetracycline, chloramphenicol, fusidic acid, fluoroquinolone, macrolide antibacterial agents, oxazolidinones, quinolone- and naphthyridone-carboxylic acid, citral, trimethoprim and sulfamethoxazole (singly and combined), aminoglycoside, polymyxin, penicillins and their derivatives. In addition, the antimicrobial may also include, for example: antifungal agents in the following classes: azoles, polyenes, echinocandins, and pyrimidines. Combinations of the any of the foregoing antimicrobial agents are also contemplated. Many of the foregoing are cationic species or their pharmaceutically acceptable salts, and in an embodiment, cationic antimicrobials are utilized in the composition. In an embodiment the composition is exclusive agents that release gas fumes, such as, for example, chlorine dioxide, or chlorine dioxide producing reactants.

In an embodiment, the barrier-forming composition does not induce mutations or the development of resistance by microbes. This is because of the mechanism of action against the microorganisms by the barrier and the selected antimicrobial.

The antimicrobial may be present, for example, in an amount ranging from about 0.0005% to 5% by weight of the total composition, such as, for example, about 0.0025% to about 1%, about 0.005 to about 0.006%, or about 0.0006% to about 0.003%. In another embodiment, the antimicrobial may be present, for example, in an amount ranging from about 0.05% to about 0.1% by weight of the total composition, such as, for example, about 0.05% to about 0.06% or about 0.06% to about 0.1%. In an embodiment, the antimicrobial is about 5% or less, or about 3% or less, or about 1.5% or less, such as when the antimicrobial used does not cause solubility problems at higher concentrations.

In embodiments, the composition may further include other components, such as, for example, copovidone and other lubricating agents, parabens such as methyl paraben or propylparaben, scenting agents, preservatives, such as sodium benzoate, buffering agents, such as monosodium and disodium phosphate, sweeteners, hydrogenated castor oil with ethylene oxide, and carboxymethylcellulose. These components may, for example, be included in amounts ranging from about 0.01% to about 5% by weight of the total composition, such as, for example, about 0.1% to about 2%. In another embodiment, the components are included, for example, in amounts of about 0.0001% to about 0.05%. Buffering agents (such as monosodium or disodium phosphate) may also be used.

Purified water and/or alcohol may be used as the diluent component of the composition. In an embodiment, the barrier-forming composition is a free-flowing liquid suitable for spraying. This is in contrast to a paste or toothpaste composition, which is typically not free-flowing and not suitable for spraying. In addition, in an embodiment, the barrier-forming composition is free of abrasives that are commonly used in toothpaste compositions.

In an embodiment, the composition consists essentially of only the gum, the humectant, and the antimicrobial, such as including only preservatives or scenting agents that do not affect the barrier or antimicrobial activity. In an embodiment, the composition is exclusive of agents for acting against the teeth and/or gums, including, for example, abrasives (such as those used in toothpastes) teeth whitening or desensitizing agents. In an embodiment, the composition is exclusive of cellooligosaccharides. In an embodiment, the antimicrobial agent is exclusive of lipids such as fatty acid ethers or esters of polyhydric alcohols or alkoxylated derivatives thereof. In an embodiment, the composition is exclusive of one or more of time-release agents, allergy-relief compounds, azelastine, silicon based oils, essential oils, polyvinyl pyrrolidone, polyvinyl alcohol, and potassium nitrate. In an embodiment, the composition is free of volatile organic compounds, including for example, volatile alcohols. In an embodiment, the composition is free of surfactant or foaming agent. For the avoidance of doubt, none of the above should be construed to mean that all embodiments are exclusive of these compounds.

In an embodiment, a method for making a barrier-forming composition includes mixing and heating the carbohydrate gum, humectant, and antimicrobial agents. In an embodiment, heating is replaced with extended mixing times. Other components may also be mixed in a single or multiple mixing steps. All components of the barrier-forming composition may be mixed at one time to produce a composition with a stable shelf life. This is in contrast to compositions that have active components that must be added separately a short time prior to use. Thus, in an embodiment, the barrier-forming composition is a stable one-part composition that does not require mixing with a second composition to activate it for use.

In an embodiment, the composition is liquid and is non-foaming.

As mentioned above, in an embodiment, the composition is suitable for spraying, and thus also has a viscosity that is suitable for spraying. In an embodiment, the composition has a viscosity of less than 500 cps such as, for example, about 490 cps to about 10 cps, or about 400 cps to about 15 cps. In another embodiment, the composition has a viscosity of about 16 to about 20 cps, such as, for example, about 17 to about 19 cps.

In an embodiment, the composition is non-toxic to humans, wherein at least a portion of the composition may be ingested and is safe for human consumption.

Without being bound by theory, the barrier-forming composition is not hydrophilic which allows the barrier-forming composition to have a greater affinity to adhere to and cover certain surfaces. Furthermore, in an embodiment, the antimicrobial being embedded in the non-hydrophilic composition will allow for sustained antimicrobial activity on treated surfaces. In an embodiment the barrier-forming composition is amphiphilic or has amphiphilic components.

One measure of hydrophilicity is the Rf (relative front) value, determined by chromatography in water. In an embodiment, the composition has an Rf value in water of 0 to about 0.25, such as about 0.0001 to about 0.15, or about 0.03 to about 0.1.

In an embodiment, the composition has a pH of about 4 to about 8, such as about 5 to about 7, or about 6 to about 7.5. In another embodiment the composition has a pH of greater than 5.5 to about 8, wherein antimicrobials such as cetylpyridinium chloride are most effective.

In general, the dual-action mechanism of providing a barrier from microorganisms and an antimicrobial agent provides a long-lasting effect, characterized by both in vitro, simulated in vivo, and in vivo examples below. In in vivo examples, the barrier-forming composition was shown to have antimicrobial effect (cidal or static) for at least 6 hours. The barrier property itself was tested in simulated in vivo tests (on artificial human mucosa EHOMs), which indicated the barrier itself had a significantly extended duration past 6 hours, such as greater than about 8 hours, about 6 to about 16 hours, and about 24 hours, or more. In addition, in vitro tests indicate the antimicrobial effect had a significantly extended duration past about 2 hours, past about 6 hours, and depending on the microorganism tested, greater than about 8 hours, about 6 to about 16 hours, and about 24 hours, or more.

Post antimicrobial effect (PAE) is defined as suppression of microbial growth that persists after limited exposure to an antimicrobial agent. Having a longer PAE is considered advantageous for antimicrobial agents as it allows for persistent inhibition of microbial growth, and may affect dosing regimens as agents with long PAEs may need less frequent administration than those with short PAEs.

In embodiments of the method and composition disclosed herein the PAE of the composition when applied to a mucosa has a PAE that persists for about 6 hours or more, such as about 6 hours to about 16 hours, or about 16 hours to about 24 hours.

As the Examples below show, the barrier-forming composition has been shown to block the passage of a wide variety of representative fungi, bacteria and viruses. Because viruses are amongst the smallest infectious microorganisms, and because the barrier-forming composition forms a mechanical barrier blocking viruses, it is expected that the barrier-forming composition would be an effective preventative treatment not only for viruses but also for larger microorganisms, including a wide range of bacteria and fungi.

Several experiments were performed to assess the safety of the composition on mammals and the ability of the spray formulation to form a protective barrier on an Engineered Human Oral Mucosa (EHOM) model. The experimental evidence showed that the composition formed a barrier over tissues, which prevents microorganisms from penetrating into the tissues

### EXAMPLES

### Example 1

Human Gingival Epithelial Cell and Fibroblast Cultures

Normal human gingival cells (epithelial cells and fibroblasts) were obtained from ScienCell Research Laboratories (Carlsbad, Calif., USA). The fibroblasts were cultured in Dulbecco's modified Eagle's medium (DME, Invitrogen Life Technologies, Burlington, ON, Canada) supplemented with fetal bovine serum (FBS, Gibco, Burlington, ON, Canada) to a final concentration of 10%. The epithelial cells were cultured in Dulbecco's modified Eagle's (DME)—Ham's F12 (3:1) (DMEH) with 5 μg/mL of human transferrin, 2 nM 3,3′,5′ of tri-iodo-L-thyronine.

0.4 μg/mL of hydrocortisone, 10 ng/mL of epidermal growth factor, penicillin and streptomycin, and 10% FBS (final concentration). The medium was changed once a day for epithelial cells and three times a week for fibroblasts. When the cultures reached 90% confluency, the cells were detached from the flasks using a 0.05% trypsin-0.1% ethylenediaminetetra acetic acid (EDTA) solution, washed twice, and resuspended in DMEM (for the fibroblasts) or DMEH-supplemented medium (for the epithelial cells).

### Example 2

Engineered Human Oral Mucosa (EHOM) Tissue

The EHOM model was produced by using the gingival fibroblasts and epithelial cells of Example 1 that were used to form a complex three-dimensional spatial cellular organization similar to that found in normal human oral mucosa. The lamina propria was produced by mixing Type I collagen (Gibco-Invitrogen, Burlington, ON, Canada) with gingival fibroblasts, followed by culture in 10% FBS-supplemented medium for four days. The lamina propria was then seeded with gingival epithelial cells to obtain the EHOM. The tissue specimens were grown under submerged conditions until the total surface of the lamina propria was covered with epithelial cells. To produce stratified epithelium, the EHOM was raised to an air-liquid interface for four more days to facilitate the organization of the epithelium into its different strata.

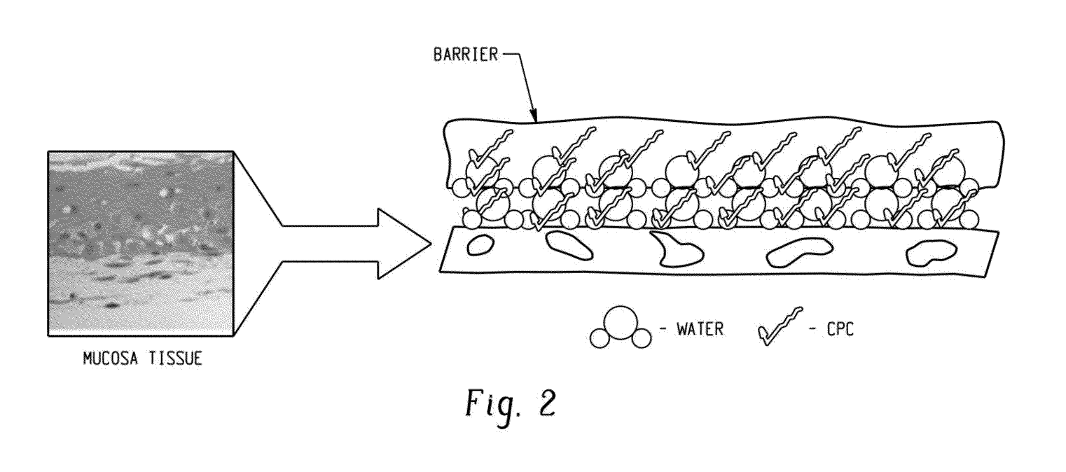

The lamina propria is a thin layer of loose connective tissue that lies beneath the epithelium and together with the epithelium constitutes the mucosa. FIG. 2 shows an illustration of the EHOM mucosal tissue, with an arrow pointing to its location in a schema depicting mucosa covered with the barrier-forming composition.

### Examples 3-9

Examples of the barrier-forming compositions were created by adding the ingredients listed below in a 50-mL centrifuge tube, and vortexing to bring to “free-flow” consistency. The constituents of the compositions and their approximate amounts are given in Table I (the values in Table I are percentages by weight of the total composition):

TABLE II

| | | | Example 5 | Example 6 | | | |
|---|---|---|---|---|---|---|---|
| | Example 3 | Example 4 | (control) | (control) | Example 7 | Example 8 | Example 9 |
| Glycerin | 7 | 35 | 35 | 35 | 35 | 7 | 7 |
| Xanthan Gum | 0.01 | 0.4 | 0.4 | 0.4 | 0.4 | 0.01 | 0.01 |
| Cetyl | 0.05 | 0.05 | | | 0.1 | 0.06 | 0.05 |
| Pyridinium | | | | | | | |
| Chloride | | | | | | | |
| Preservatives | No | No | No | Yes | Yes | Yes | Yes |

*Purified water comprised the remaining portion of the composition.
**Preservatives included methylparaben (0.1%), propylparaben (0.1%), sodium benzoate (0.5%)

Based on the results below, the preservatives were found to be superfluous to the barrier formation and antimicrobial activity.

### Example 10

Monolayer Wound Repair Assay

Wound repair assays were performed on the epithelial cells and fibroblasts of Example 1. Briefly, gingival epithelial cells (1×10⁴ cells) and fibroblasts (1×10³ cells) were seeded into wells of 6-well plates and grown in Dulbecco's modified Eagle's (DME)—Ham's F12 (3:1) (DMEH) with 5 μg/mL of human transferrin, 2 nM 3,3′,5′ of tri-iodo-L-thyronine, 0.4 μg/mL of hydrocortisone, 10 ng/mL of epidermal growth factor, penicillin and streptomycin, and 10% FBS (final concentration). Upon confluency, wounds were made in the confluent monolayer of each well using a 200 μL pipette tip.

### Examples 11-15

In Examples 11 and 12, the epithelial cell cultures from Example 10 were exposed to diluted barrier-forming compositions of Examples 3 and 4 for about 2 minutes. In Examples 13 and 14, the fibroblast cultures from Example 10 were exposed to diluted barrier-forming compositions of Examples 3 and 4 for about 2 minutes. Prior to exposure, Example 3 was diluted with saline to a 1% concentration and Example 4 was diluted with saline to a 5% concentration. Following exposure, the spray was washed out twice with warm sterile culture medium, then cell cultures were over layered with DME for fibroblasts and DMEH for epithelial cells, and cultured for 6 and 24 hours in a 5% CO₂ humid atmosphere at 37° C. Control Example 15, which was an untreated culture from Example 10, was also tested.

Wound repair/cell migration was ascertained using an optical microscope, and digital photographs were taken (Nikon, Coolpix 950). The percentages of wound closure (cell growth/migration) were calculated by comparing relative wound areas before and after exposure to our barrier spray using formula I:

$$1. \frac{\begin{matrix}( initial scratch size ) - \\ ( size after a specified culture period )\end{matrix}}{value of the ( initial scratch size ) \times 100} .$$

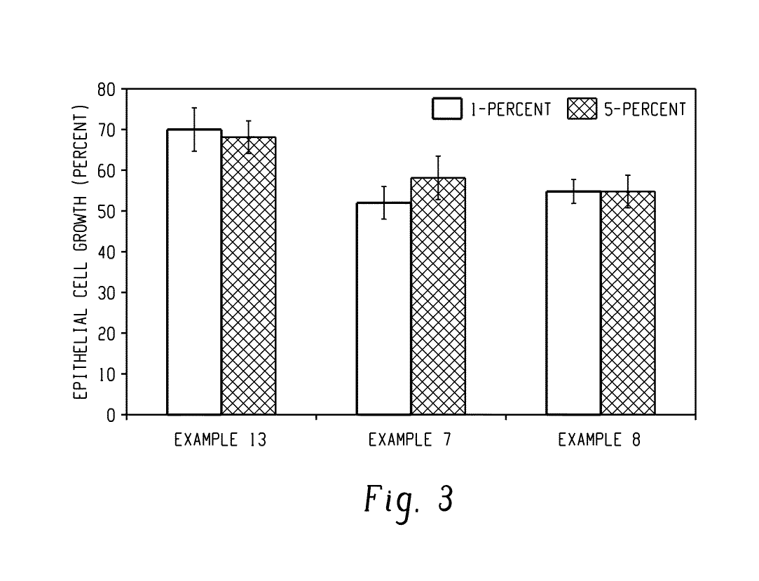

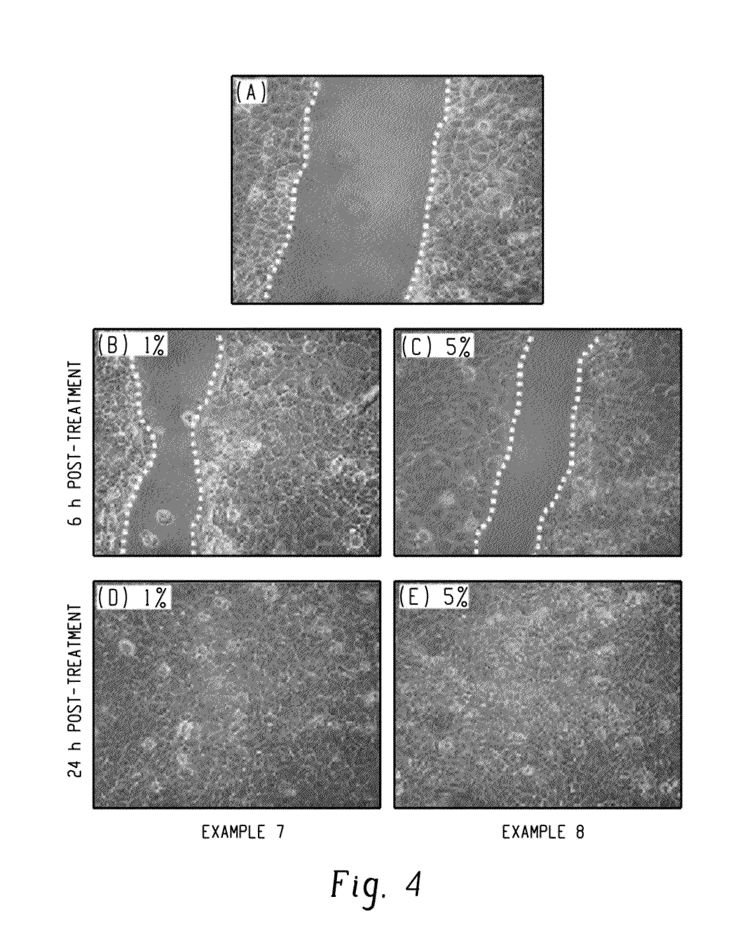

FIG. 3 shows a graph of the wound repair data generated from formula I after 6 hours post-treatment. FIG. 4 also shows photographs indicating the wound repair of the epithelial cells cultures treated with the 1% diluted barrier-forming composition and the control Example 15.

Following contact with the barrier-forming compositions, epithelial cells (FIG. 4A) and fibroblasts (data not shown) migrated progressively starting at 6 hours post contact with the barrier-forming compositions of Examples 11 and 12. Epithelial cells were small and cuboidal in shape, in both non-exposed and barrier-forming composition-exposed cultures (FIG. 4A-E). Fibroblasts were elongated cells with small nucleus in both culture conditions (unexposed and cetylpyridinium chloride-based products exposed cells) (data not shown). Cell growth and migration to cover the wounded zone were comparable in both the control Example 15 and the treated Examples 11 and 12 as compared to the original wound (FIG. 4F). Follow up over 24 hours showed that each of Examples 11 and 12 covered the edges of the scratch giving confluent monolayers (FIG. 4D-E).

This experiment was repeated five separate times with similar results. The treated Examples 11 and 12 did not show a major side effect on gingival epithelial cells growth/migration nor on cells differentiation (no cells presenting large cytoplasm and large nucleus).

### Examples 16-19

Cytotoxicity Assay

The engineered human oral mucosa (EHOM) model of Example 2 was used to determine whether the composition of Examples 10 and 11 were safe and did not promote tissue damage or cell necrosis. In Example 16 and 17, the epithelium surface (10 cm²) was over layered with a thin layer (300 μl) of the Example 3 barrier-forming composition at a dilution of 5% and the Example 4 composition at a dilution of 1% (both were diluted in serum free culture medium) for time periods of about 2 minutes. The variation in time period was not deemed to have an effect on the results and just reflects the time it took to conduct the procedures. Control Example 18 was a control that was not treated with the barrier-forming composition. The EHOM tissues were then rinsed twice with warm sterile culture medium and incubated in a 5% CO₂ humid atmosphere at 37° C. for 24 hours. Following this incubation period, to assess whether the engineered tissue was damaged, each EHOM was macroscopically examined for the presence of holes due to the contact with the barrier spray formulation. Photos were taken of these EHOM to confirm such possibility. Additionally, biopsies were collected from each EHOM and subjected to histological staining using eosin and hematoxylin.

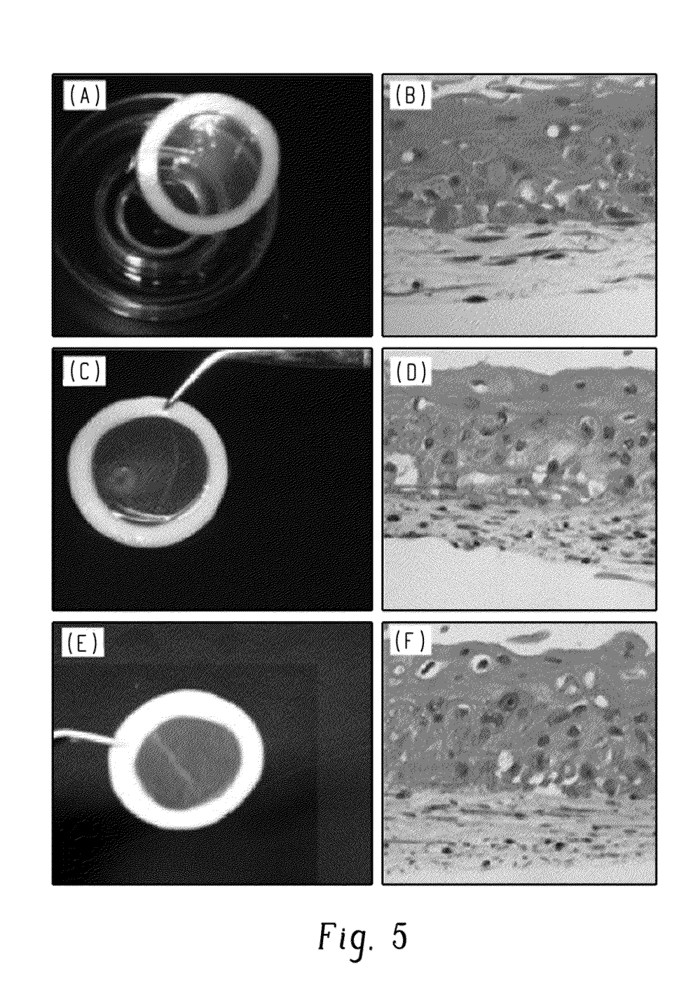

In Examples 16 and 17, EHOM exposed to Examples 3 and 4 in the 1 and 5% dilutions, similar to untreated control Example 18, do not show any macroscopic damage such as holes. (See FIG. 5). The absence of such damage was monitored after 10 minute tissue contact with 5% dilution Example 3 mouthwash (Example 7A) and 1% dilution spray (Example 7B) compositions. This was confirmed by histological analyses showing multilayer organisation of the epithelium on the top of a fibroblast-populated connective tissue. No specific damage was observed in EHOM tissues in Examples 16 or 17. The Example 16 and 17 treated-EHOM structures were comparable to the untreated control Example 18.

The same observation was noted in an Example 19, which was the same as Example 16, except it included an EHOM treated with a 1% dilution (data not shown).

FIG. 5 shows the effect of the barrier-forming composition on EHOM macroscopic shape and structure. Panels A and B show control Example 18. Panels C and D show Example 16 (spray composition) and panels E and F show Example 17 (mouthwash composition).

### Examples 20-25

Side effects, if any, of the barrier spray composition on EHOM injury was also assessed by measuring the leakage of intracellular LDH into the culture medium.

In Examples 20-23, EHOM tissues were exposed to (A) 1% or (B) 5% dilutions of Examples 3 and 4, respectively, for 10 minutes, followed by growth in culture medium for 24 hours. Examples 24 and 25 were controls that were not treated. 50 μl of a supernatant of each of Examples 20-25 were then transferred to a sterile 96-well flat-bottom plate. Each well was supplemented with 50 μl of reconstituted substrate mix, and the plate was incubated for 30 min at room temperature in the dark. To estimate LDH levels, aliquots of the culture supernatant were collected and subjected to an LDH cytotoxicity assay (Promega, Madison, Wis., USA), as per the manufacturer's protocol. This assay measures the conversion of L-lactate and NAD to pyruvate and NADH by the released LDH. To stop the reaction, 50 μl of stop solution was added to each well. Next 100 μl of the mixture were transferred to a 96-well flat-bottom plate, and the absorbance was read at 490 nm with an X-Mark microplate spectrophotometer (Bio-Rad, Mississauga, ON, Canada).

In the LDH and wound repair experiments, the following test methods were used. Each experiment was performed at least four times, with experimental values expressed as means±SD. The statistical significance of the differences between the control (absence of barrier spray) and the test (presence of spray) values was determined by one-way ANOVA. Posteriori comparisons were done using Tukey's method. Normality and variance assumptions were verified using the Shapiro-Wilk test and the Brown and Forsythe test, respectively. All of the assumptions were fulfilled. P values were declared significant at ≦0.05. Data were analyzed using the SAS statistical package (version 8.2, SAS Institute Inc., Cary, N.C., USA).

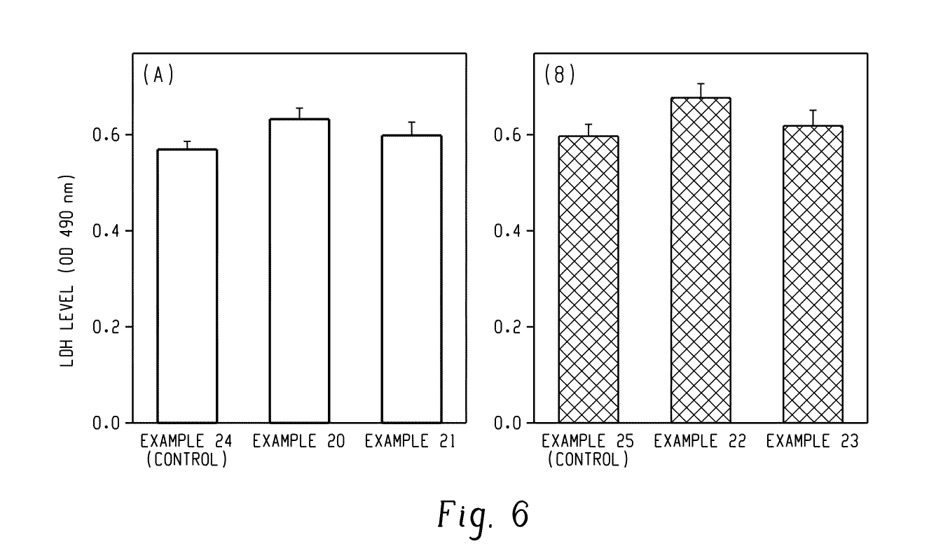

Results presented in FIG. 6 showed no significant difference in LDH levels in barrier-forming composition treated structures as compared to untreated tissues. These observations were made at 6 hours (data not shown) and 24 hours (FIG. 6) post-exposure to the barrier-forming composition treated Examples. FIG. 6(A) shows Control Example 24 and the 1% dilution treated Examples 20 and 21; and FIG. 6(B) shows a Control Example 25, and the 5% dilution treated Examples 22 and 23. This data confirm the non-toxic effect of Examples 3 and 4. Similarly, contact for 2 minutes with Examples 3 and 4 did not show any difference as compared to the controls (data not shown).

### Example 26

Determination Whether Barrier-Forming Composition Damages EHOM Structure.

EHOMs of Example 2 were treated with Example 4 for about 2 minutes, washed with culture medium then cultured for 24 hours. Tissue was then examined for possible macroscopic tissue damage (presence or not of holes). Tissue damage was also investigated by histological analyses. For this purpose, biopsies were taken from each EHOM. They were fixed with 4% paraformaldehyde solution and then embedded in paraffin. Thin sections (4 μm) were stained with eosin-hematoxilyn. Sections were mounted with a coverslip in 50%-glycerol mounting medium, observed through an optical microscope, and photographed. No damage to the treated EHOMs was ascertained.

### Examples 27 and 28

Determination Whether the Barrier-forming composition Affects Mechanical Barrier Function of EHOM Against Microbial Passage Through Mucosal Tissue.

In Examples 27 and 28, two approaches were used to determine whether the control Examples formed a barrier that blocked the microbial passage through the mucosal tissues and also had an inherent anti-microbial effect. Growth in pass-through chamber and growth on EHOM surface was assessed by evaluating growth in agar media.

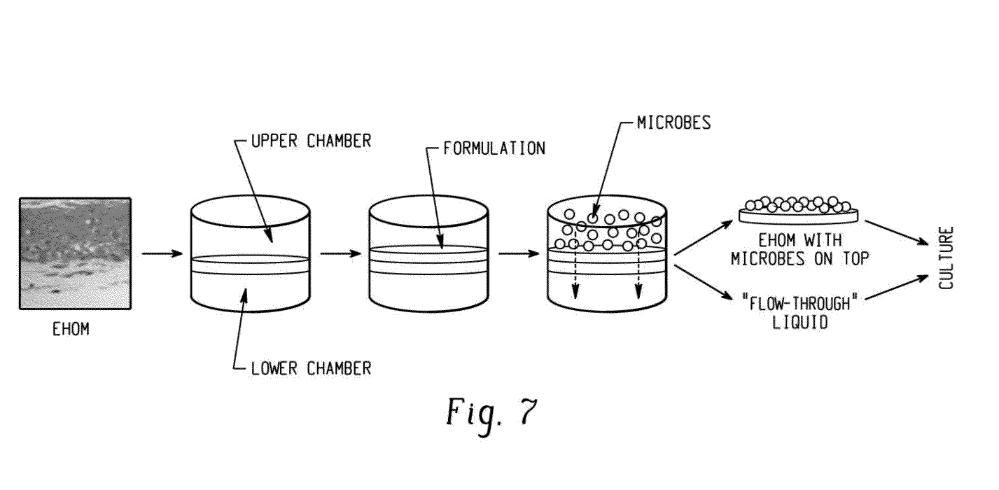

In Example 27, EHOMs of Example 2 were put in contact with 1 and 5% dilutions (diluted in serum free culture medium) of Example 4 for 2 minutes. Tissues were then washed twice with serum free culture medium then over layered with 1×10⁶ candida microbial cells in a volume of 300 μl. Tissues were then put on air-liquid culture plates and incubated for 24 hours in 5% CO₂ humid atmosphere at 37° C. Next, the culture medium underneath the EHOM (ventral chamber) was collected and seeded on Sabouraud agar plate to verify whether or not the microorganisms penetrated through the tissue and reached the culture medium below. A culture was also obtained from the EHOM surface and seeded on Sabouraud agar plate. The process is graphically depicted in FIG. 7.

In Example 28, EHOMs of Example 2 that were treated with 1 and 5% dilutions of the Example 4 composition for 2 minutes were over layered with candida microbial cells for 24 hours were flipped onto Sabouraud dextrose agar plates and left in place for 5 minutes. The EHOMs were then removed and the plates were incubated for 24 hours at 30° C., after which microbial growth was ascertained macroscopically and photographed. Each experiment was repeated 5 independent times with similar results.

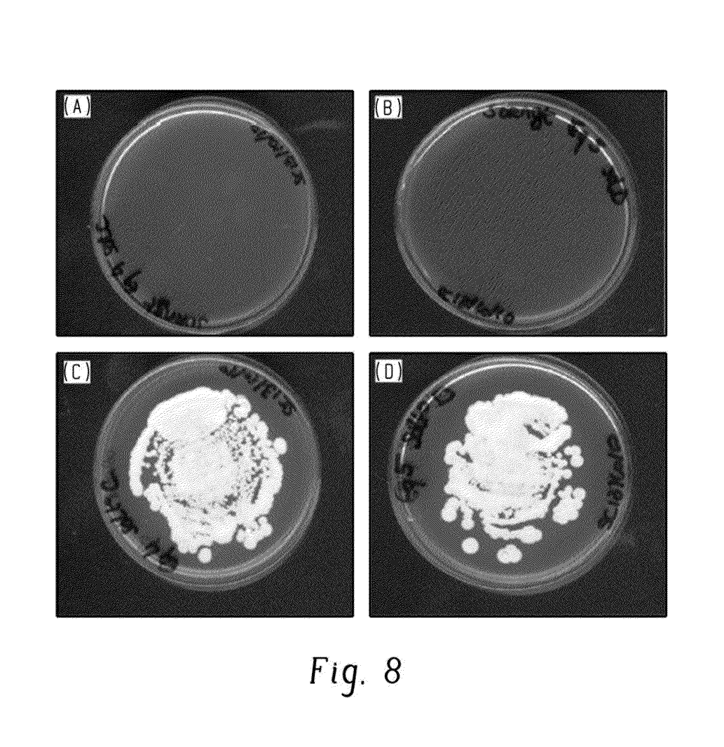

FIG. 8 shows the results of the cultures of the EHOM surface (panels C and D) and the culture of the pass-through liquid from the bottom (ventral) chamber (panels A and B). The A and C panels were EHOMs treated with a 1% dilution of Example 4, and the B and D panels were EHOMS treated with a 5% dilution of Example 4. This data indicates that Example 4 composition forms a barrier that prevents passage of microbes through the EHOM tissues but does not have an inherent anti-microbial effect.

### Examples 29 and 30

In Examples 29 and 30, Examples 27 and 28 were repeated, except the EHOM were infected with S. mutans. Similar results were obtained that indicated that the barrier-forming compositions formed a barrier preventing the S. mutans microbes from passing through the barrier, but did not have an antimicrobial effect.

### Examples 31 and 32

Determination Whether the Barrier-Forming Composition Affects Mechanical Barrier Function of EHOM Against Microbial Invasion.

In Example 32, a set of EHOM tissues from Example 2 was treated with the barrier-forming composition of Example 4 and then overlaid with C. albicans. In control Example 31 a control set was not treated with the barrier-forming composition prior to overlayering with C. albicans. Immediately after each contact period, biopsies were taken from each EHOM, fixed with paraformaldehyde solution, and embedded in paraffin. Thin sections (4 μm) were stained with eosin-hematoxylin. Sections were observed using an optical microscope to analyze the invasion/penetration of microbial cells into the tissue. Following microscopic observations, representative photos were taken from each condition and presented. The experiment was repeated three times with similar results. Similar results were also obtained with treatment with Example 3 (data not shown).

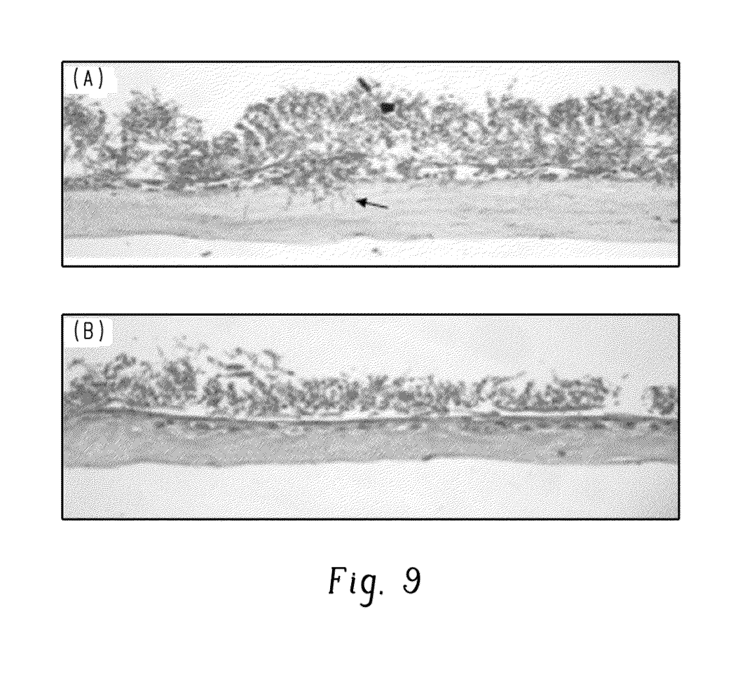

FIG. 9 shows the effect of the barrier-forming composition on microbial invasion of EHOM tissues. Panel (A) is a representative photograph of the untreated control Example 31, and panel (B) is a photograph of the treated Example 32. The arrow indicates invading fungal hyphae in the untreated control Example 31.

### Examples 33-40

The EHOM model described above was also used to evaluate the ability of Examples 5-7 to form a barrier that: (a) prevents oral bacteria (S. mutans) and fungi (Candida albicans) from penetrating/invading human oral mucosa, and (b) does not cause damage to host cells (cytotoxicity assay).

Examples 33-40 were formulated according to Table III below.

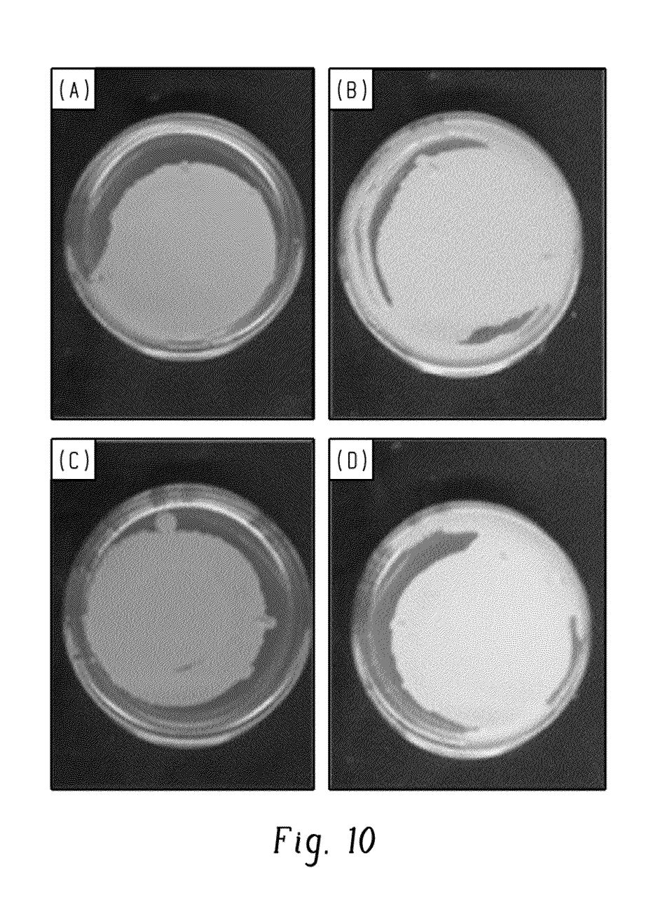

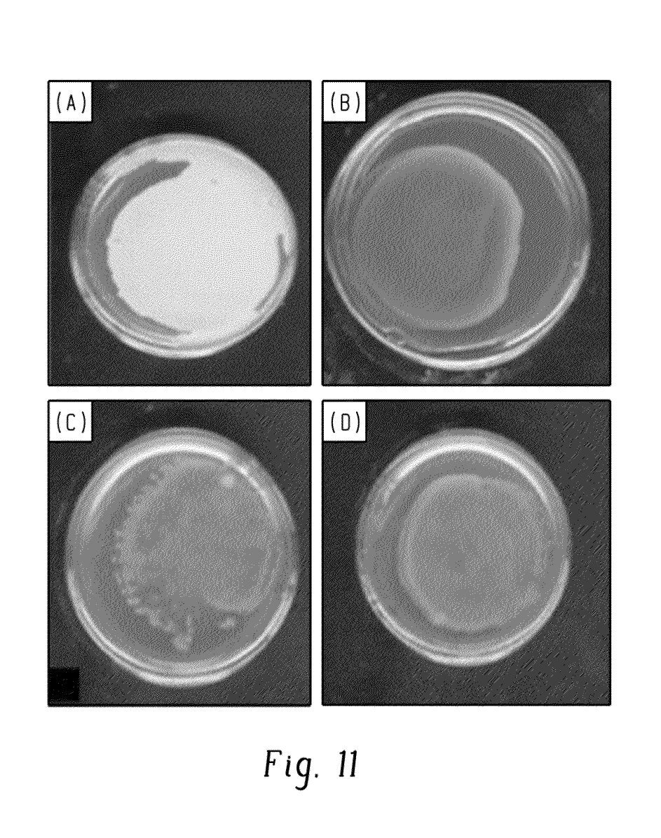

TABLE III

| | Barrier-forming | | |
|---|---|---|---|
| | composition Pre- | | |
| | Treatment | Microbe Overlay | FIG. reference |
| Example 33 | None | C. albicans | FIG. 10(A) |
| Example 34 | Example 5 | C. albicans | FIG. 10(B) |
| Example 35 | Example 6 | C. albicans | FIG. 10(C) |
| Example 36 | Example 7 | C. albicans | FIG. 10(D) |
| Example 37 | None | S. mutans | FIG. 11(A) |
| Example 38 | Example 5 | S. mutans | FIG. 11(B) |
| Example 39 | Example 6 | S. mutans | FIG. 11(C) |
| Example 40 | Example 7 | S. mutans | FIG. 11(D) |

In Examples 33-40, after pre-treatment and incubation according to the procedures of Examples 27 and 28: (1) the flow-through medium was collected from the lower chamber; and (2) tissues were flipped and placed onto the surface of Sabouraud dextrose agar Petri dishes, and incubated for 24 hours. Collected flow-through media were spread onto agar media plates, and incubated for 24 hours also as described in Examples 27 and 28. Table III also indicates the Figure in which a photo of each Example was taken showing the microbial growth on each flipped Example culture.

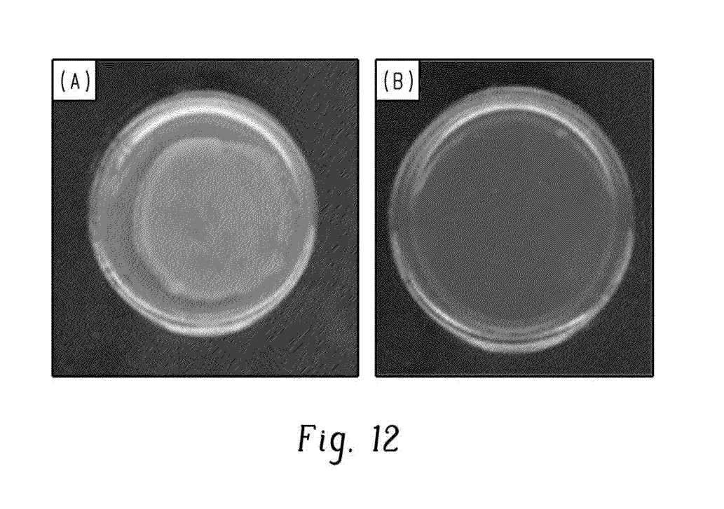

FIGS. 10 and 11 show that both Candida and Streptococcus were able to grow on the surface of EHOM treated with the compositions of Examples 5-6. In contrast, as shown in FIG. 12, no microbial growth was observed when the “flow-through” medium collected from the lower chambers of EHOMs of Examples 36 or 40, i.e. those treated with the Example 7 composition. This indicates that treatment of the EHOMs with the Example 7 composition did not cause damage to the surface of the mucosal tissues and organisms were unable to penetrate the treated EHOM. Similar results were obtained with EHOM treated with the compositions of Examples 5 and 6 (data not shown). These data indicate that the combination of glycerine and xanthan gum is capable of forming a protective barrier on mucosal tissues.

### Examples 41-47

Tested Formulations are not Toxic and do not Cause Damage to the Cells/Tissues

In Examples 41-47, the EHOM model was used to assess the toxicity of the composition. Examples 41-47 were formulated as stated in Table IV.

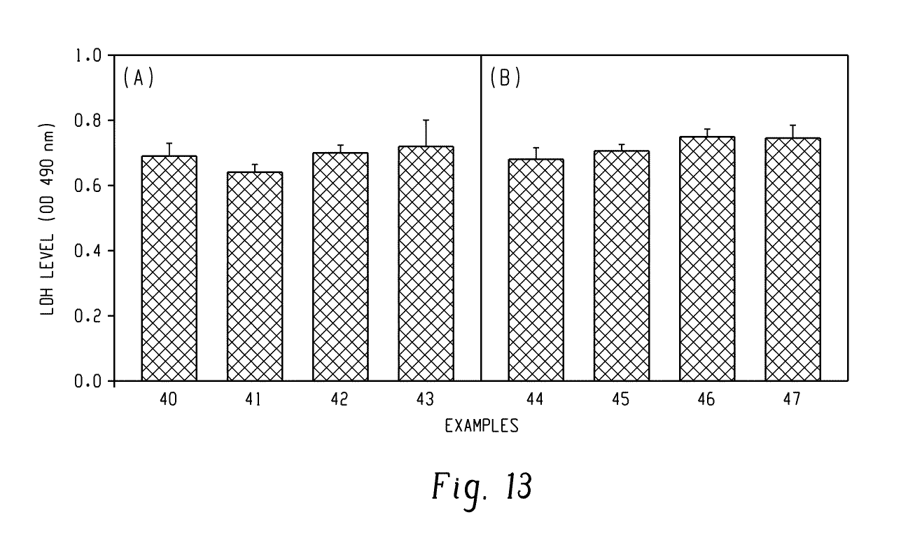

TABLE IV

| | Barrier-forming | | |
|---|---|---|---|
| | composition Pre- | | |
| | Treatment | Microbe Overlay | FIG. Reference |
| Example 41 | None | C. albicans | FIG. 13(A) |
| Example 42 | Example 5 | C. albicans | FIG. 13(A) |
| Example 43 | Example 6 | C. albicans | FIG. 13(A) |
| Example 44 | Example 7 | C. albicans | FIG. 13(A) |
| Example 41A | None | S. mutans | FIG. 13(B) |
| Example 45 | Example 5 | S. mutans | FIG. 13(B) |
| Example 46 | Example 6 | S. mutans | FIG. 13(B) |
| Example 47 | Example 7 | S. mutans | FIG. 13(B) |

After pre-treatment and incubation according to the procedures of Examples 27 and 28, culture supernatant was collected from the Example 41-48 EHOM tissues and used to measure LDH activity.

As shown in FIG. 13, no significant increase in LDH levels was observed in Examples 41-48 irrespective of whether the formulations contained cetylpyridinium chloride with or without preservatives and infected with either Candida albicans or S. mutans, respectively. These data confirmed the non-toxic effect of the Example barrier-forming compositions and that these formulations maintained the integrity of the host mucosal tissues.

Data are mean±SD and were computed as stated in Example 25 above. No significant difference between untreated and treated tissues was noted.

Taken together, the data indicates that the example compositions represent an effective and a safe barrier that can prevent microorganisms from penetrating and invading human mucosal tissues.

### Examples 48-61

Preclinical evaluation of the barrier-forming composition showed that the composition was effective against many bacteria and yeasts. The antimicrobial activities of the Example 7 barrier-forming composition were evaluated against a number of clinical isolates obtained from patients, including S. salivarius, P. gingivalis, S. pyogenes, S. pneumonia, Fusobacterium nucleatum, S. mutans, S. aureus, Y enterocolitica, S. oxalis, S. mitis, C. albicans, C. krusei, C. tropicalis, and C. glabrata. Activity of the Example 7 barrier-forming composition was evaluated by determining its minimum inhibitory concentration (MIC) using reference methods described in the Clinical and Laboratory Standards Institute (CLSI) documents M07-A8, M11-A7, and M27-A3.

A standardized inoculum of several types of aerobic or anaerobic bacteria (1×10⁴ cells/ml) was incubated with serially diluted solutions of Example 7 (containing 0.1% CPC, or 1 μg/ml) or 2% chlorhexidine gluconate (CHX, 20 μg/mL) as a comparative example. Cells were allowed to grow in the presence or absence (growth control) of the test agents for 24 hours. The MIC for each agent was defined as the concentration that induced a 100% growth inhibition (compared to no-drug control).

A similar microdilution-based CLSI method (M27-A2) was used to evaluate the activity of Example 7 against albicans and non-albicans Candida species.

TABLE V

| | | Example 7 | |
|---|---|---|---|
| | | MIC | Chlorhexidine MIC |
| | Organism | (μg/mL CPC) | (μg/mL chlorhexidine) |
| Example 48 | S. salivarius | 0.98 | 19.6 |
| Example 49 | P. gingivalis | 0.98 | 19.6 |
| Example 50 | S. pyogenes | 0.98 | 19.6 |
| Example 51 | S. pneumonia | 0.98 | 19.6 |
| Example 52 | F. nucleatum | 1.95 | 19.6 |
| Example 53 | S. mutans | 1.95 | 19.6 |
| Example 54 | S. aureus | 3.91 | 19.6 |
| Example 55 | Y. enterocolitica | 3.91 | 19.6 |
| Example 56 | S. oralis | 500 | 19.6 |
| Example 57 | S. mitis | 500 | 19.6 |
| Example 58 | C. albicans | 0.25 | 19.6 |
| Example 59 | C. krusei | 0.06 | 19.6 |
| Example 60 | C. tropicalis | 0.06 | 19.6 |
| Example 61 | C. glabrata | 0.125 | 19.6 |

The barrier-forming composition was also found to have potent antimicrobial activity against: MRSA, Acinetobacter baumannii, Streptococcus sanguis, S. gordonii, and Aggregatibacter actinomycetemcomitans.

As can be seen in Table V, the Example 7 composition exhibited potent activity against many aerobic and anaerobic bacteria, as well as the fungi.

The MIC of the Example 7 barrier-forming composition against S. oralis and S. mitis was noticeably elevated (500 μg/mL) compared to other organisms. It is interesting to note that S. oralis and S. mitis are normal commensals of the oral cavity. Activity of the commonly used antimicrobial chlorhexidine (2% solution) was also determined by the same method. Table V shows the MIC of the Example 7 barrier-forming composition and chlorhexidine (2% solution) as a comparative example against various microorganisms.

Taken together, these results demonstrate that Example 7 possesses potent activity against pathogenic bacteria and fungi commonly isolated from the oral cavity. This activity was more potent than that observed for chlorhexidine.

A similar activity profile was observed for the barrier-forming compositions of Examples 10 and 11.

### Example 62

As a further comparison, published data shows that the tested barrier-forming composition has a better or at least equivalent MIC compared to CPC alone (i.e. not in a composition according to the barrier formulation disclosed herein). See Frank-Albert Pitten and Axel Kramer, “Efficacy of Cetylpyridinium Chloride Used as Oropharyngeal Antiseptic,”Arzneim.-Forsch./Drug Res. 51 (II), pp 588-595 (2001), which is incorporated herein by reference. The data varies based on the microorganism tested, but, for example, CPC (alone) against S. mutans has an MIC of 5.0-6.25 μg/mL, which is much less effective than the 1.95 μg/mL reported in Example 53. This was an unexpected result since CPC has the risk of losing its activity when mixed with other excipient chemicals in a formulation. See Department of Health and Human Services (Food and Drug Administration) (1994) Oral Health Care Drug Products for Over-the-Counter Human Use; Tentative Final Monograph for Oral Antiseptic Drug Products. Proposed Rules (21 CFR Part 356, Docket No. 81N-033A, RIN 0905-AA06). Federal

Register 59:6084-124.

### Examples 63-69

Duration of Antimicrobial Activity of Barrier-Forming Compositions In Vitro: Determination of Post-Antimicrobial effect (PAE)

The PAE of Example 8 against several microorganisms was evaluated in Examples 63-68. Control Example 69 was also provided. Several microorganisms were exposed to Example 8 (at a concentration equal to the MIC) for 1 min followed by three washes to remove residual formulation. The treated cells were then spread on agar medium plates, which were incubated at 37° C., and the time taken for the cells to regrow was determined. PAE was expressed as the time (in hours) for which growth inhibition (%) was maintained by the Examples 63-68, compared to the untreated control Example 69.

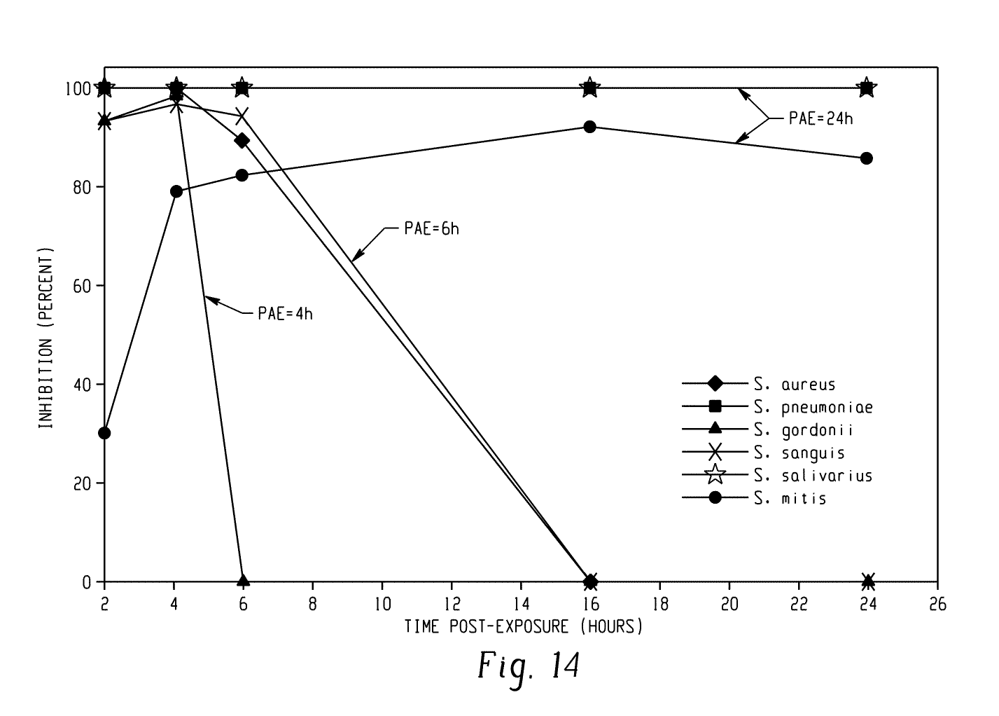

As shown in FIG. 14, Example 8 exhibited a PAE ranging between 4 hours to 24 hours, depending on the organism tested (S. aureus, S. pneumonia, S. gordonii, S. sanguis, S. salivarius, and S. mitis). Similar activity of Example 8 was observed against Candida (data not shown). Other Example barrier-forming compositions exhibited similar PAE against microorganisms.

### Example 70

Testing of PAE for the Example 7 barrier-forming composition against S. mutans compared to a similar comparative Example with lower CPC content of 0.7% showed that the PAE of Example 7 was 24 hours, while that of Comparative Example 70 was 6 hours. Thus demonstrating that Example 7 exhibits greater prolonged antimicrobial activity than comparative Example 70, and that additional amounts of CPC have more than a simple additive effect on anti-microbial activity.

### Examples 71-76

Scanning electron microscopy was also used to show that treatment of S. sanguis, (Example 71), S. oralis, (Example 72), and C. albicans (Example 73) with the composition of Example 3 resulted in destruction of cellular integrity.

In Examples 71-73, cells were grown in the presence of Example 3 for 24 hours. Next, the cells were washed to remove residual formulation, dehydrated by passing through a series of alcohol solutions (10% to 100%, v/v) and processed for SEM analysis. Control Examples 74-76 differed from Examples 71-73 in that they were not grown in the presence of Example 3.

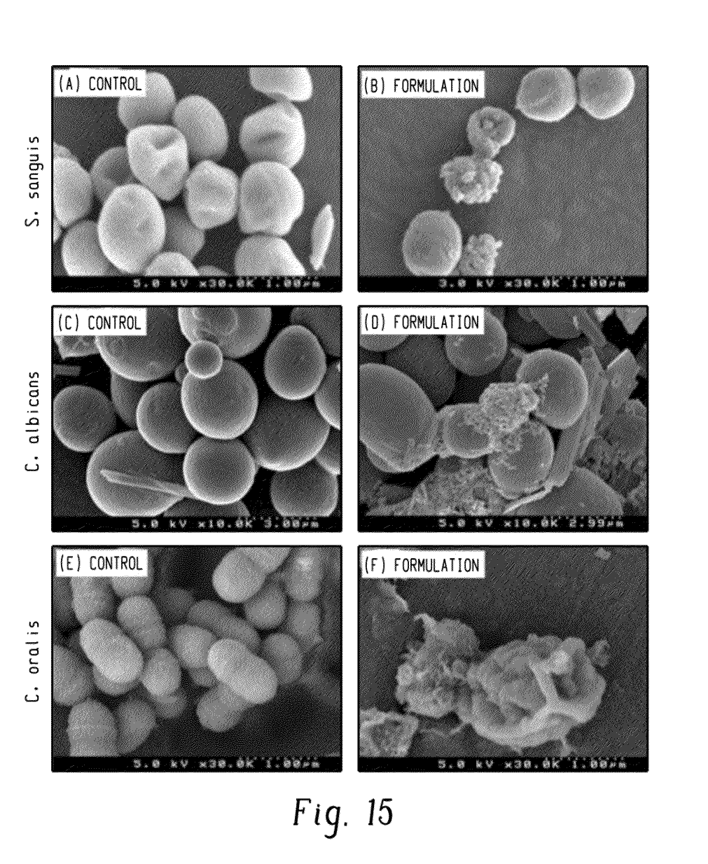

The SEM photos showed that unlike untreated control Examples 74-76, which demonstrated healthy intact cells (FIG. 15 A, C, E), microbes exposed to the Example 3 barrier-forming composition were deformed, collapsed, and exhibited total destruction of cellular integrity with clear evidence of leakage of cytoplasmic material. (FIG. 15, B, D, F).

### Examples 77-79

Since biofilms are precursors to certain infectious diseases, in Examples 77-79, experiments were performed to determine whether the barrier-forming compositions can prevent formation of biofilms by bacteria and yeasts. Biofilms were formed using an in vitro model. See Chandra et al. “In vitro Growth and Analysis of Candida Biofilms” Nature Protocols 3(12): 1909-1924 (2008).

In Examples 77-79a standard biofilm model was employed to determine whether the Example 3 barrier-forming composition exhibits activity against bacterial and fungal biofilms. In Examples 77-79, three different microorganisms (C. albicans, S. oralis, and S. salivarius) were adhered on substrate for 90 minutes to allow biofilms to form to adhesion phase. Next, discs containing the adherent bacteria were incubated for 15, 30 or 60 minutes with 50% concentration of Example 3 (1:1 dilution with appropriate medium). Following incubation, biofilms were scraped, spread on culture media, incubated and colony forming units (CFUs) were determined. Media diluted with phosphate buffered saline (PBS, 1:1) were used as a control. Table VI reports data at 0 (Control), 15, 30, and 60 minutes.

TABLE VI
Effect of Barrier-forming composition on Early Phase Biofilms
(log CFU)

| | Example 77 | Example 78 | Example 79 |
|---|---|---|---|
| Exposure time | C. albicans | S. oralis | S. salivarius |
| Control | 5.44 | 3.25 | 3.16 |
| 15 min | 0 | 0 | 0 |
| 30 min | 0 | 0 | 0 |
| 60 min | 0 | 0 | 0 |

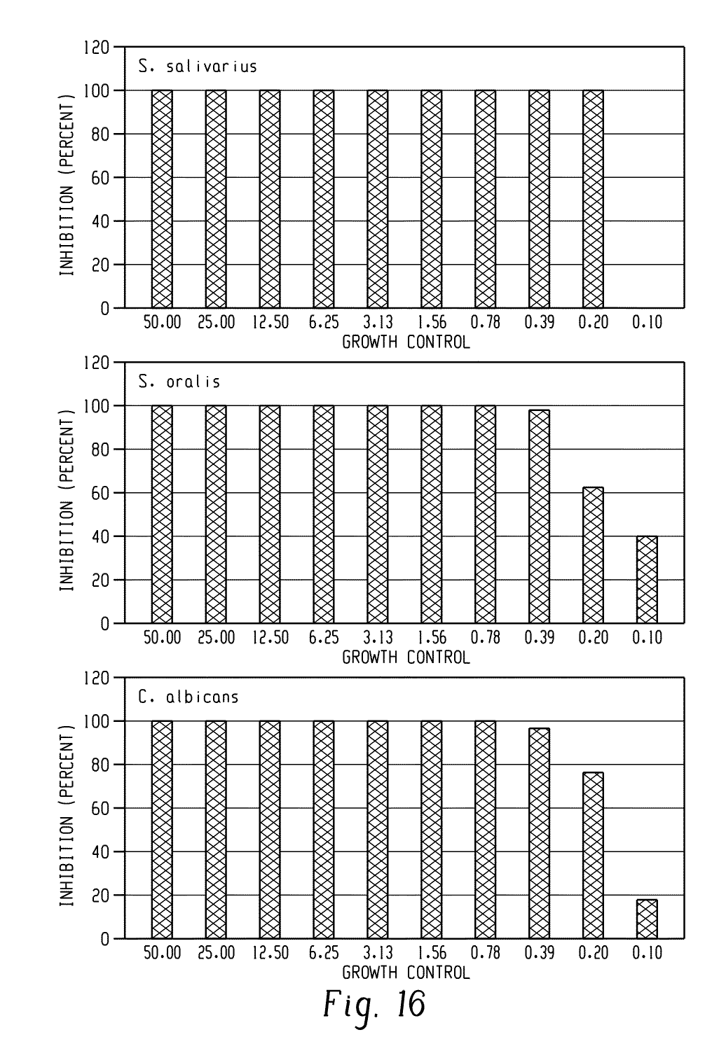

FIG. 16 also reports data on Examples 77-79 as a graph of % inhibition versus growth control. These results showed that Example 3 barrier-forming composition inhibited bacterial and fungal microbes with an MIC of 0.2% against biofilms formed by S. salivarius, S. oralis, or C. albicans.

### Examples 80 and 81

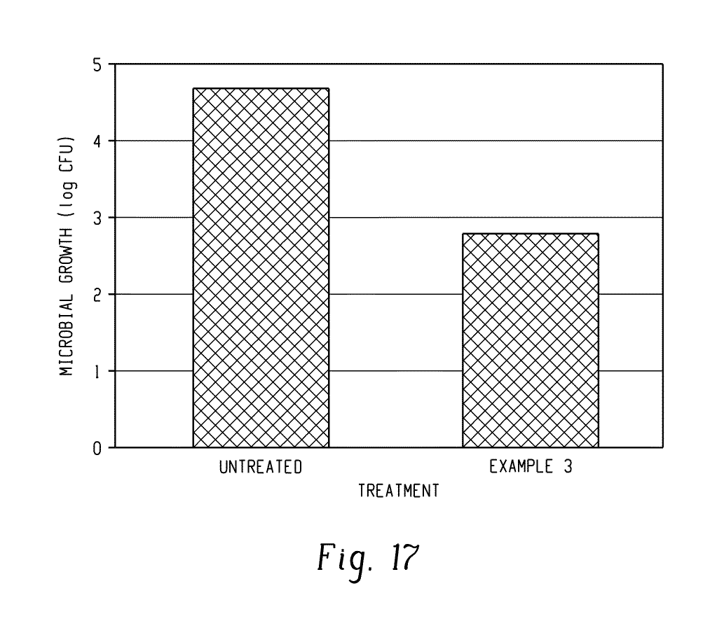

In Example 80 we evaluated the effect of 1 minute exposure of C. albicans early phase biofilms to Example 3, and found that even with an exposure for as short a time as 1 minute, it was able to inhibit biofilm formation (FIG. 17). Example 81 was an untreated control sample.

### Examples 82-84

Ability of Barrier-Forming Composition to Treat Mature Biofilms

To determine whether the barrier-forming composition can treat biofilms, we evaluated its activity against fully formed mature biofilms. Biofilms were grown to mature phase, and then exposed to Example 7 for 2 or 4 hours, and the resulting CFUs were determined. A composition that causes at least 2-log reduction in microbial CFUs compared to untreated cells is considered to be effective against microbial biofilms.

As shown in Table VII, exposure to Example 7 resulted in complete eradication of biofilms formed by C. albicans and S. oralis, and a 3.4-log reduction in CFUs for biofilms formed by S. salivarius compared to the untreated control (log CFU=3.95 vs. 7.36, respectively).

TABLE VII
Effect of Example 7 on mature biofilms (log CFU)

| | Example 82 | Example 83 | Example 84 |
|---|---|---|---|
| Exposure time | C. albicans | S. oralis | S. salivarius |
| Control | 5.60 | 7.40 | 7.36 |
| 2 h | 0 | 0 | 4.00 |
| 4 h | 0 | 0 | 3.95 |

In summary, the results indicate that Example 7 possesses potent activity against biofilms formed by bacteria and fungi.

### Examples 85-86

The Barrier-Forming Composition is also Active Against Viruses

The activity of barrier-forming composition against viruses, including respiratory viruses (influenza virus H1N1, strain 2009/H1N1/infA) and the human immunodeficiency virus (HIV) was determined.

The barrier-forming composition inhibits the infectivity of influenza A

To evaluate the effect of the barrier-forming composition on the infectivity of influenza virus, Madin Darby canine kidney (MDCK) cells were grown to >90% confluence at 37° C. prior to infection. MDCK cells are used routinely for assays involving influenza viruses.

In Example 85 cell monolayers were exposed to the Example 7 barrier-forming composition. In control Example 86 the cell layers were exposed to optiMEM (+P/S,+Lglu) tissue culture media for different times: (1) T1: 30 min exposure, (2) T2: 1 h exposure, (3) T3: 2 h exposure. Next, the formulation was removed and the cell monolayers were infected with influenza virus (multiplicity of infection (MOI)=0.1). Cells that were untreated or infected immediately after exposure (TO) were used as baseline controls. Infected cells were then centrifuged, resuspended in 500 μL of growth medium, and incubated at 32.5° C. for 48 hours. Immunofluorescence microscopy (using FITC labeled anti-influenza antibody) was also used to evaluate the effect of the Example 7 barrier-forming composition on the ability of influenza virus to infect mammalian cells.

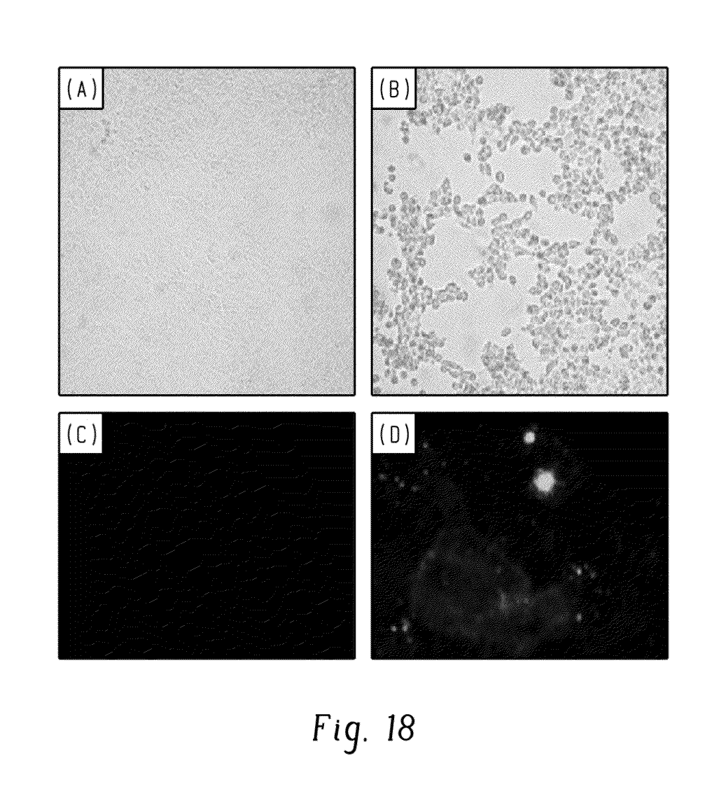

FIG. 18 shows the effect of Example 7 on cytopathic effects of influenza-infected MDCK cells (Example 85) (panels A and C), and control Example 86 (panels B and D). Images were obtained from: phase contrast (A-B), and immunofluorescence microscopy (C-D). No identifying cytopathic effect (CPE) was observed in formulation-treated cells. Untreated cells displayed typical CPE including focal rounding and degenerative changes.

The data showed that exposure of cell monolayers to Example 7 for 30 minutes, 1 hour, or 2 hours remained confluent and healthy (Example 85). In contrast, in the untreated cells and cells treated immediately prior to infection (TO) (control Example 86) demonstrated substantial cytopathic effect. As seen in FIG. 18 panel C, no fluorescence was observed in the barrier-forming composition treated cells of Example 85, while the untreated cells of Example 86 exhibited fluorescence (FIG. 18 panel D).

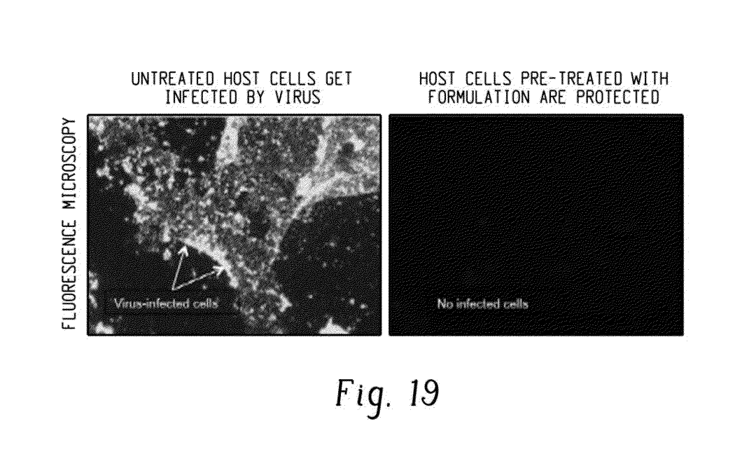

Further fluorescence microscopy images corresponding to Examples 85 and 86 are presented in FIG. 19.

### Examples 87 and 88

Activity of Barrier-Forming Composition on Viral Load Using Quantitative PCR.

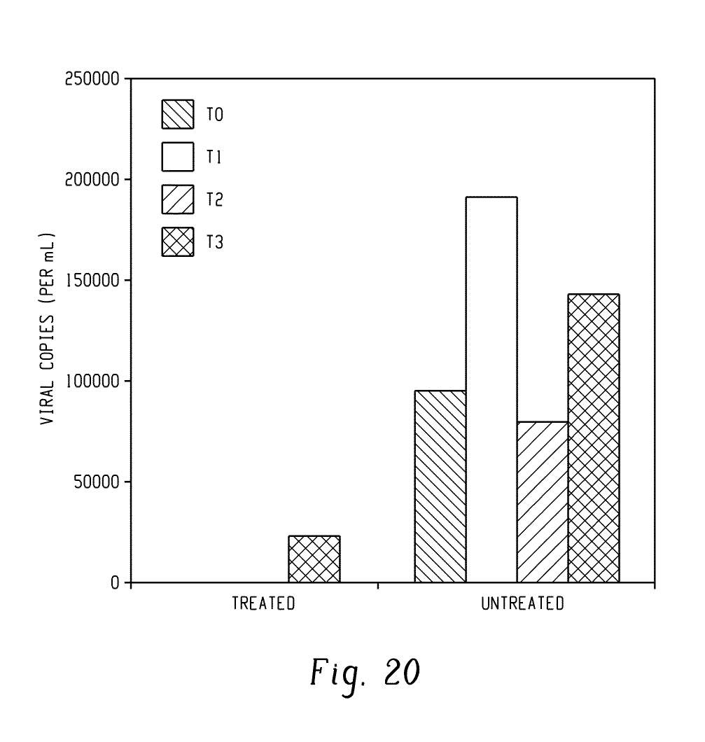

FIG. 20 shows levels of influenza virus in infected treated cells (Example 87) and untreated cells (Example 88), as determined by quantitative PCR. In Example 87, cells were treated with Example 7 and in control Example 88 the cells were left untreated. Later the supernatants were collected and analyzed for the presence of virus.

Cell culture supernatants from the same assay as in Examples 87 and 88 were collected and nucleic acid extracted using QIAamp Viral RNA Kit (QIAGEN, Valencia, Calif.). Random hexamer primers (Invitrogen Carlsbad, Calif.) were used to create a cDNA library for each specimen. Reverse transcription reactions were performed with M-MLV RT (Invitrogen, Carlsbad, Calif.) according to the manufacturer's specifications. Quantitative analysis was performed on a StepOne Plus Taqman Real Time PCR (Applied Biosystems, Branchburg, N.J.) using TaqMan Universal PCR Master Mix (Applied Biosystems, Branchburg, N.J.), 2 μl of cDNA sample, and primers/probes targeting the influenza matrix gene. A reference standard was prepared using a cDNA fragment of the H1N1 matrix gene and human RNAse P amplified by conventional RT-PCR, gel purified (QIAquick, Qiagen, Valencia, Calif.), and quantified using a spectrophotometer (Beckman Coulter, Brea, Calif.).

As shown in FIG. 20 and Table IV, the Example 87 cells treated Example 7 for 30 min or 60 min did not have detectable influenza at 48 hours post infection. Moreover, treatment with Example 7 for 2 hours resulted in a 6-fold decrease in viral load, compared to the untreated control or those treated immediately prior to infection (Example 88).

TABLE VIII

| | Example 87 | Example 88 (control) |
|---|---|---|
| 30 min | 0 | 192000 |
| 60 min | 0 | 79800 |
| 120 min | 23400 | 143000 |

### Examples 89-91

Barrier-Forming Composition has Direct Antiviral Effect Against Influenza Virus

To determine whether the barrier-forming composition has direct antiviral activity against influenza virus, we infected African Green Monkey Kidney (CV-1) cells (grown in 24-well plates to 90% confluence) with influenza virus that was pre-treated with Example 7. CV-1 cells are routinely used a highly susceptible substrate for diagnosis and study of viruses.

In Examples 89-91, a standardized amount of influenza (0.1 MOI) was pretreated for 5 minutes at room temperature with: (1) Example 7 (to form Example 89), (2) control Example 6, a compound without CPC but with preservatives (to form Example 90), and (3) control Example 5 placebo alone (a compound without CPC and preservatives) (to form Example 91). After the 5 minute incubation virus/drug mix was diluted by an additional equal volume with optiMEM (+P/S,+Lglu) to dilute out the treatment compositions.

In Examples 89-91, CV-1 cells were prepared as described in above. The Example 89-91 treated and untreated viruses were then inoculated onto the cells as described above.

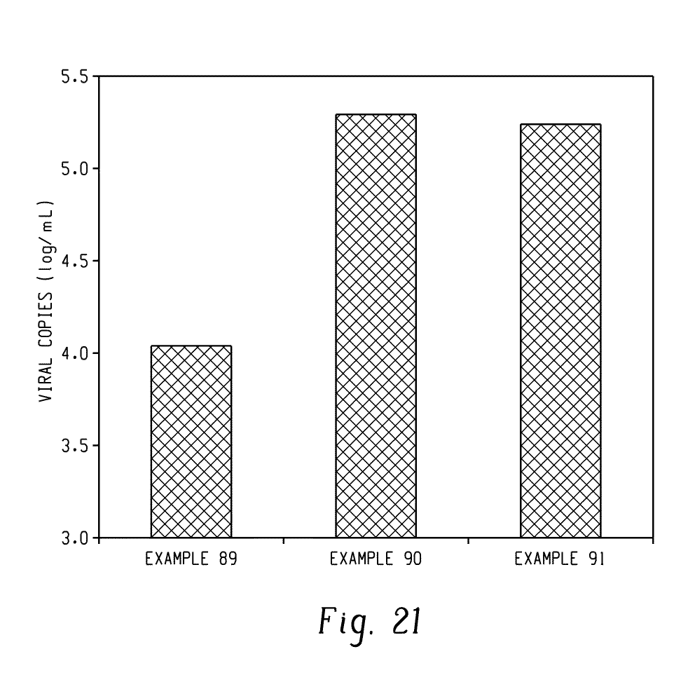

Influenza viral load was determined by real time PCR as described above. The data as shown in FIG. 21 showed significant decrease in viral load for influenza virus pretreated with the Example 7 barrier-forming composition containing the antimicrobial agent CPC (Example 89), compared to those containing only the barrier-forming composition and/or preservative but no CPC (Examples 90 and 91). Pre-treatment of virus with Example 7 exhibited significant decrease in viral copies, compared to formulations with no CPC.

These results demonstrate that the Example 7 barrier-forming composition possesses direct antiviral activity against influenza virus that is not inherent in Examples 5 and 6.

### Examples 92 and 93

In Examples 92 and 93, the barrier-forming composition's ability to inhibit the infectivity of influenza A (2009/H1N1/infA) was tested. African Green Monkey Kidney (CV-1) cells were grown in 24-well plates to 90% confluence. Next, the barrier-forming composition, Example 7, was applied to the cells (20% Example 7, 80% OptiMeM, working CPC concentration of 0.02%.) in Example 92. Each time point matched with control Example 93 (No barrier-forming composition applied, 100% OptiMeM). The barrier-forming composition was allowed to dwell on the surface for 30 minutes, and then removed from the ceil monolayer. Cells were thoroughly washed twice with sterile optiMEM (+PfS,+Lglu). Influenza was inoculated at MOi=0.1 at 30 minute intervals from T0 through T+6 hours. Following infection, cells were then centrifuged @ 2200 rpm×30 minutes and 500 μl of optiMEM (+P/S, +Lglu, 2 μg/ml trypsin (sigma-Aldrich, St Louis, Mo.)) was applied. Infected cells were grown at 32.5° C. for 96 hours at 5% CO₂. The influenza viral load was determined by real time PCR.

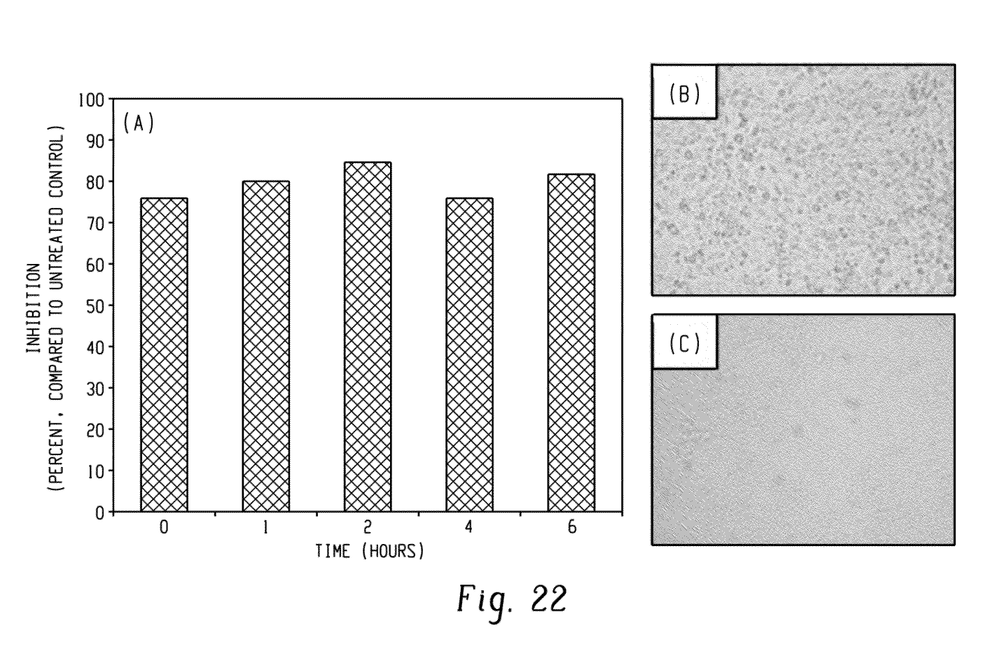

As shown in FIG. 22, pre-treatment of host monolayers with glycerine-xanthan gum formulation results in inhibition of viral infection by up to 84.93% compare to untreated controls. The fact that inhibition of viral infection was observed in host cells despite removal of the barrier-forming composition demonstrates that the barrier-forming composition formed a protective barrier on host cells, which prevented viral invasion for at least 6 hours.

FIG. 1B may be referred to as a possible mechanism accounting for the inhibition of infection.

### Examples 94-96

Barrier-Forming composition Exhibits Activity Against HIV

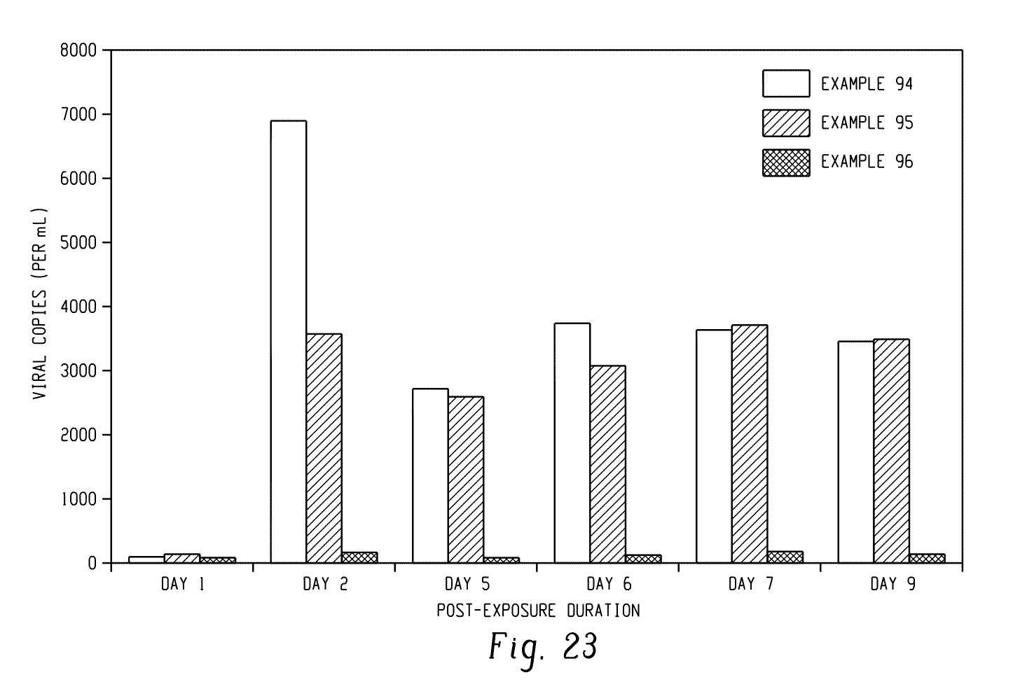

Examples 94-96 determined whether the barrier-forming composition possessed activity against HIV. Host MT mammalian cells were plated into 96-well round bottom plates at a density of 15,000 cells/well in RPMI/10% FBS/PS. The next day (Day 2), virus was pretreated with control Example 5 (to form Example 94), control Example 6 (to form Example 95), or Example 7 (to form Example 96) for 5 minutes and added to cells. After 24 hours of exposure to formulation, the MT (macaque) mammalian cells were washed 3 times with phosphate buffered saline (PBS) and fresh media was replaced. Supernatant (10 μL) was collected post-treatment on Days 1, 2, 5, 6, 7, and 9, and the viral load was determined by reverse transcriptase (RT) activity. FIG. 23 shows a graph of the viral copies per mL for each of Examples 72-74 over a 9 day span.

The results showed that Example 7 in Example 96 exhibited anti-HIV activity at all time points monitored post-treatment.

The control Example 5 or control Example 6 without CPC and/or preservative in Examples 94 and 95 exhibited only minimal anti-HIV activity.

In summary, our findings demonstrate that the barrier-forming composition Example 7 containing CPC exhibits long-lasting antiviral activity against HIV.

### Example 97

Representative organisms viral lesions are important infections in different mucosal tissues. In Example 97 an experiment was performed to determine whether the barrier-forming composition exhibits activity against the common oral Epstein-Barr virus (EBV). Western blotting was used to evaluate the ability of the Example 8 barrier-forming composition to degrade lytic viral protein EAD (indicating inhibition of viral replication).

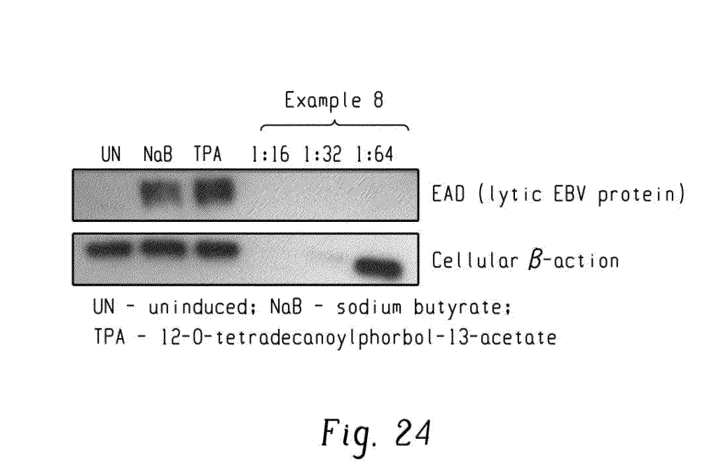

In Examples 97, EBV-infected gastric epithelial cells were exposed to different dilutions (1:16, 1:32 and 1:64) of Example 8, and the presence of EAD protein was detected using specific antibodies. Presence of cellular β-actin was used as an indicator of epithelial cell integrity. As shown in FIG. 24, 1:64 dilution of Example 8 degraded EAD without affecting cellular actin. These results demonstrate that Example 8 specifically inhibits viral replication, and as such, is an effective anti-viral and useful for prevention of viral infection.

### Examples 98-100

Duration of Anti-Microbial Barrier Versus Commercial Mouthwash Product

To determine the duration for which the barrier-forming composition can maintain the antimicrobial activity, bacteria and fungi were exposed to an EHOM of Example 2 that was treated with the barrier-forming composition of Example 7 in a well and an EHOM of Example 2 that was treated with a comparative commercial product in a well for 2 minutes. The bacterial and fungal microbes were overlaid on top of the control untreated EHOM (Example 98) and the treated EHOMs (Example 99 and Comparative Example 100). Next the residual (flow-through) solution was removed from the bottom well (lower chamber of the EHOM model) and spread onto agar medium plates. FIG. 7 depicts this test method for further clarity. These plates were then incubated at 37° C., and the number of microbial cells (colony forming units, CFUs) growing after 24 hours were counted.

In control Example 98 an untreated EHOM was tested. In Example 99 S. mitis bacteria was overlaid on the barrier-forming composition as described above. Example 100 is a comparative example showing the activity of commercially available LISTERINE (containing ethanol (26.9%), menthol, thymol, methyl salicylate, and eucalyptol) against S. mitis bacteria. Table IX shows the results.

TABLE IX

| | CFUs of S. mitis bacteria in flow | | |
|---|---|---|---|
| | through liquid from EHOM | | |
| Time post- | Example 98 | | Example 100 |
| exposure | (control) | Example 99 | (comparative) |
| 2 hours | 1150000 | 5820 | 780000 |
| 4 hours | 1400000 | 5500 | 800000 |
| 6 hours | 1600000 | 6000 | 840000 |

### Examples 101-103

In Examples 101-103, the same procedure of Examples 98-100 was performed except Candida albicans fungus was tested on the barrier-forming composition as described above. Table X shows the results. Example 103 is comparative, showing the activity of commercially available LISTERINE.

TABLE X

| | CFUs of Candida albicans in flow | | |
|---|---|---|---|
| | through liquid from EHOM | | |
| Time post- | Example 101 | | Example 103 |
| exposure | (control) | Example 102 | (comparative) |
| 2 hours | 1150000 | 12000 | 124000 |
| 4 hours | 2900000 | 12000 | 252000 |
| 6 hours | 3900000 | 13000 | 350000 |

The data further showed that Example 7 barrier-forming composition maintained activity for up to and including 24 hours. Taken together, these results showed that unlike LISTERINE, the Example 7 barrier-forming composition continued to maintain an intact barrier on EHOM tissues for up to and including 24 hours.

### Examples 104-153

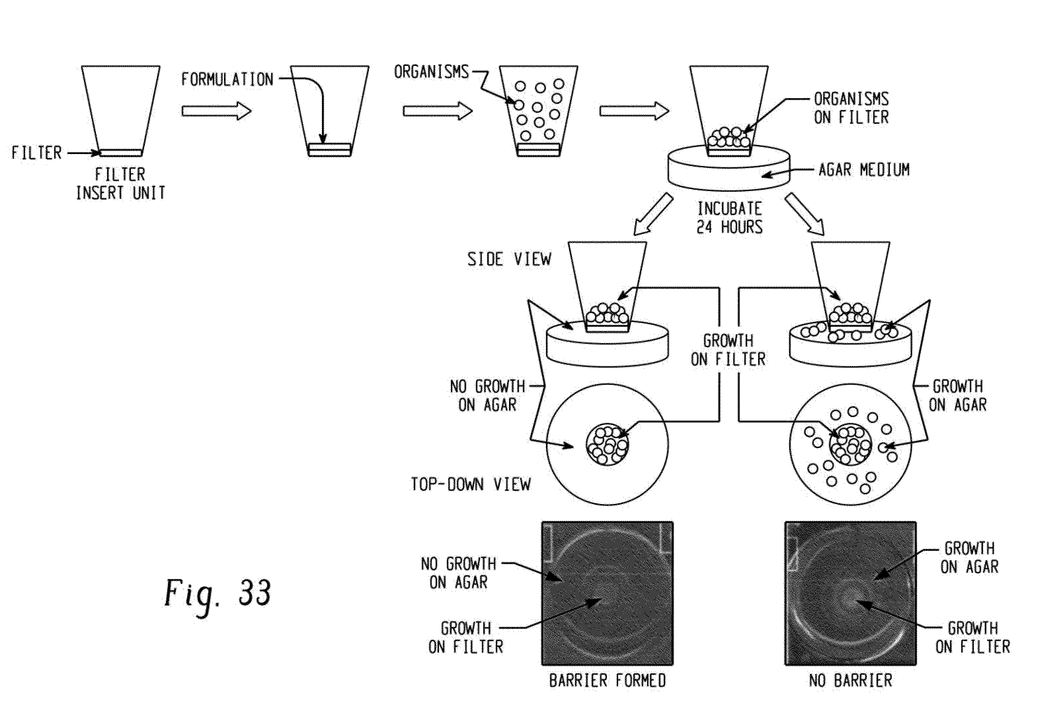

To identify further examples of concentrations of glycerin and xanthan gum that can form a barrier effective in preventing the passage of microorganisms, different concentrations of the gum xanthan gum and humectant glycerin were tested (5%-95% glycerin; 0.005%-0.5% xanthan gum) singly and in combination using an in vitro filter insert-based barrier model. FIG. 33 shows the general test method used for Examples 104-153.

Filter inserts of 3 μm and 8 μm diameter pore size were used for testing the passage of bacteria (Streptococcus salivarius) and fungi (Candida albicans), respectively. Glycerin or xanthan gum or their combinations (100 μL aliquots) were overlaid on the surface of the filter to form a barrier. The filter had a diameter of 24 mm. Thus, the film had a thickness of approximately 0.01 mm on the filter, mimicking a value in the range of thicknesses of the composition film when applied in a therapeutically effective amount to the mouth. Next, 5×10⁴ cells of either bacteria or fungi were applied on top of the formed barrier in the filter inserts. Next, we placed these filter inserts on the surface of agar medium (Brain Heart Infusion (BHI) medium for bacteria, Sabouraud Dextrose (SD) medium for fungi) in 6-well plates. The plates along with the filter inserts were incubated overnight for 24 hours at 37° C.

The plates were monitored for the presence of bacterial or fungal growth CFUs (colony forming units) in the agar medium as well as in the filter insert. Microbial growth in the filter insert only, but not in the agar medium, demonstrated that an effective barrier was formed on the filter, which prevented passage of microorganisms. Conversely, growth in the agar medium around the filter insert suggested that the tested agents failed to form an effective barrier, allowing the organisms to go through the filter.

The results reported in Table XI showed that glycerin was able to form a barrier at concentrations greater than or equal to 55%, when tested alone. In contrast, xanthan gum alone did not form a barrier at any of the concentrations tested (ranging from 0.005% to 0.4%). However, it was observed that when combined with 0.01% xanthan gum, a barrier was formed at glycerin concentrations 7%, 45%, 55%, and 65%. Furthermore, combination of 0.4% xanthan gum with glycerin concentrations of 7%, 15%, 25%, 35%, 45%, 55%, and 65% also formed a barrier. Therefore, specific combinations of glycerin and xanthan gum were identified that can form a barrier that prevents passage of microorganisms in an in vitro filter insert-based model.

TABLE XI

| | | Examples | Example | Examples | Examples | Examples | Examples | Examples |
|---|---|---|---|---|---|---|---|---|
| | | 104-112 | 113 | 114-121 | 122-129 | 130-137 | 138-145 | 146-153 |
| | | Xanthan Gum (%) | | | | | | |
| | | 0 | 0.005 | 0.01 | 0.05 | 0.1 | 0.2 | 0.4 |
| Glycerine (%) | 0 | No | No | No | No | No | No | No |
| | 5 | No | | No | No | No | Yes | No |
| | 7 | No | | Yes | | | | Yes |
| | 15 | No | | No | No | No | Yes | Yes |
| | 25 | No | | No | No | No | Yes | Yes |
| | 35 | No | | No | No | No | Yes | Yes |
| | 45 | No | | Yes | No | No | Yes | Yes |
| | 55 | Yes | | Yes | No | No | Yes | Yes |
| | 65 | Yes | | Yes | No | No | Yes | Yes |

‘No’: no barrier formed;
‘Yes’: barrier formed

Microbial cells retained by the compositions of Examples 104-153 formed on filter inserts were trapped by the barrier, and were viable, thus demonstrating that the formed barrier does not have an inherent antimicrobial property without an antimicrobial agent. In other words, the microbes retained in the barrier were still active and could pose a threat to infection; for example, if they are freed from the barrier by abrasion or after the barrier loses its integrity.

It should be noted that an effective barrier may be formed at lower concentrations of glycerine and/or xanthan gum when an effective antimicrobial is added. This is because the antimicrobial and barrier act in tandem to stop and/or kill the harmful microbes.

### Examples 154-157

In addition to the Examples above on EHOM samples, further testing was performed to further demonstrate the barrier-forming composition (a) does not damage the host tissues, and (b) is able to prevent microbial invasion into the human mucosal tissues. These criteria were tested for two representative combinations (glycerine:xanthan gum; 7%:0.01% and 35%:0.4%), selected based on the in vitro results of Examples 104-153, because they successfully formed a barrier. Formulations containing these two combinations were tested using the EHOMs of Example 2 that mimic the mucosal lining.

The EHOMs were treated with the various formulations of Examples 3 and 4 in either 1% (Examples 154 and 155) or 5% (Examples 156 and 157) dilutions in normal saline [0.9%]) for about 2 minutes to form Examples 154-157. Each EHOM was covered with 300 μL of one of the tested formulations and left under the sterile hood for 10 minutes at room temperature (25° C.). At the end of the contact period, tissues were washed twice with culture medium to remove the formulations.

### Example 158

After treatment with Examples 3 and 4, the EHOMs were then examined for possible macroscopic tissue damage (presence of holes). Tissue damage was also investigated by histological analyses. For this purpose, biopsies were taken from each EHOM and fixed with 4% paraformaldehyde solution and then embedded in paraffin. Thin sections (4 μm) were stained with eosin-hematoxilyn and mounted with a coverslip in 50%-glycerine mounting medium, observed through an optical microscope, and photographed. The treated EHOMs and a control untreated EHOM were examined for macroscopic tissue damage and structural changes. There was no visible damage (holes) in the untreated or treated tissues at a magnification of ×500 in five different EHOMs tested.

### Example 159

Evaluation of Barrier-Forming Composition Causing Damage to Host Cells (Cytotoxicity Assay).

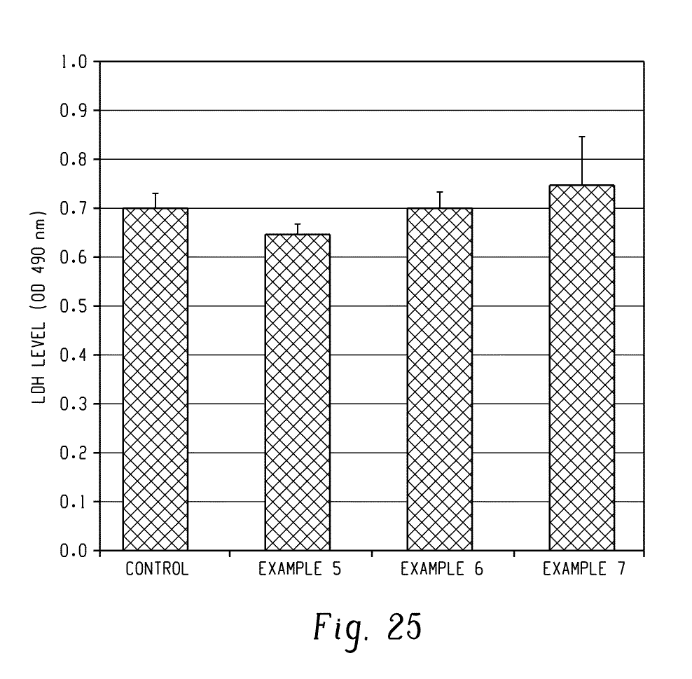

The Example 156 and 157 EHOMs that were treated with the 5% dilution of Examples 3-7 of the barrier-forming composition were then over-layered with 1×10⁶ cells of either C. albicans or S. mutans in a volume of 100 μl. The EHOMs were then placed on air-liquid culture plates and incubated for 24 hours in 5% CO₂ humid atmosphere at 37° C. Following this incubation period, aliquots of the culture supernatant were collected and subjected to a Lactate dehydrogenase (LDH) cytotoxicity assay (Promega, Madison, Wis., USA), as per the manufacturer instructions. 50 μl of each supernatant were transferred to a sterile 96-well flat-bottom plate. Each well was supplemented with 50 μl of reconstituted substrate mix, and the plate was incubated for 30 minutes at room temperature in the dark. To stop the reaction, 50 μl of stop solution was added to each well. Next 100 μl of the mixture was transferred to a 96-well flat-bottom plate, and the absorbance was read at 490 nm with an X-Mark microplate spectrophotometer (Bio-Rad, Mississauga, ON, Canada). LDH was assessed using LDH cytotoxicity assay. Data are means±SD. No significant difference between EHOMs treated with the Example composition and an untreated, uninfected EHOM control was noted. FIG. 25 graphically shows the results of these tests on an untreated control example, and those treated with Examples 5, 6, and 7. Similar results were obtained for the EHOMs treated with Examples 3 and 4. The tested Examples all maintained the integrity of the mucosa.

### Example 160

Effect of Barrier-Forming Composition Formulations on Gingival Cell Growth/Migration

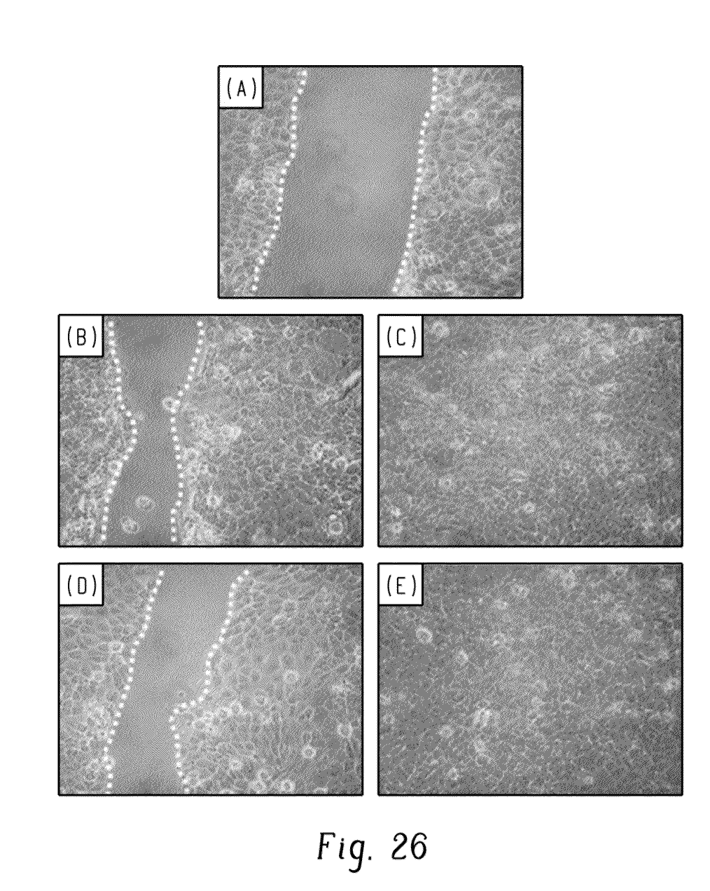

Wound repair assays were performed. Briefly, oral (gingival) epithelial cells (1×10⁴) and fibroblasts (1×10³) were seeded into wells of 6-well plates and grown in appropriate culture medium. Upon confluency, wounds were made in the confluent monolayer of each well using a 200 μL pipette tip. Cultures were then exposed to 1 and 5% by weight dilutions of Examples 3 and 4 for about 2 minutes. Following exposure, the formulations were washed out twice with warm sterile culture medium, and then cell cultures were over layered with DEM for fibroblasts and with DEM for fibroblasts and DMEH for epithelial cells, and cultured for 6 and 24 hours in a 5% CO₂ humid atmosphere at 37° C. Wound repair/cell migration was ascertained using optical microscope, and digital photographs were taken (Nikon, Coolpix 950). FIG. 26 shows representative photographs of a wounded oral epithelial cell culture treated with Example 3 (5% dilution) for 10 minutes, immediately after the wound (panel A), after about 6 hours (panel D), and after about 24 hours (panel E). Panels B and C show an equivalent wound on an untreated control confluent culture of oral epithelial cells after about 6 hours and about 24 hours, respectively. Similar results were seen for the cell culture treated with Example 4.

The percentage of wound closure (cell growth/migration) was calculated by comparing relative wound areas before and after exposure to the formulations using formula I stated above.

The epithelial cells were small and cuboidal in shape in both treated and untreated cultures. Similar results were observed for scratch wound model using fibroblasts (data not shown). Taken together, this data indicate that example compositions are not toxic and do not negatively impact cell growth/migration and wound healing.

### Example 161

Glycerine-Xanthan Gum Formulations Form a Coating on the Human Oral Mucosa

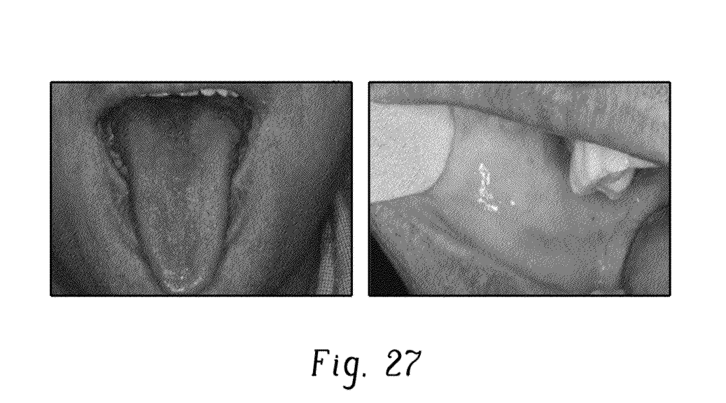

To determine whether glycerine-xanthan gum formulation can form a coating on the human oral mucosa, we spiked the Example 7 formulation with Gentian Violet (GV) as a marker dye. The spiked product (750 μL) was sprayed onto the oral cavity of human volunteers. Post-application, the oral cavity was inspected for staining, and the images were captured using a digital camera. As shown in FIG. 27, the formulation stained both cheeks and the dorsal/ventral surface of the tongue.

### Examples 162 and 163

Exposure of Microbes to Barrier-Forming Composition Inhibits Cell Growth: Time-lapse Microscopy

To determine the inhibitory activity and duration for which barrier-forming compositions exhibit activity against microbes, time-lapse analysis was performed on cells exposed to the barrier-forming composition, compared to untreated bacteria and fungi.

In Example 162, S. mutans microbial cells were exposed to Example 7 for one minute, washed to remove any residual agent, and allowed to grow in a petri-dish containing fresh growth medium. Growth of organisms at 37° C. was monitored for a 6 hour period, and photomicrographs were taken every 20 minutes over the 6 hour incubation period using a camera connected to the microscope.

In control Example 163 the same procedure was followed with untreated cells.

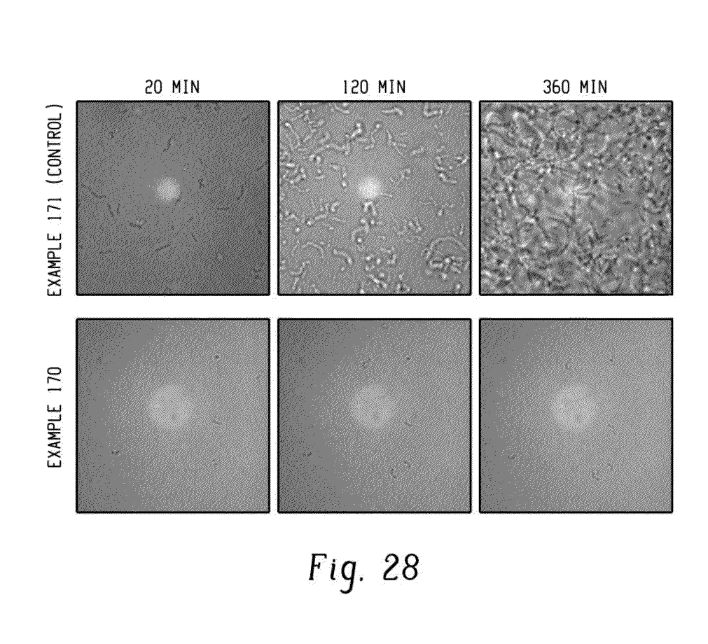

As shown in FIG. 28, in contrast to the untreated bacteria, where cells reached confluence by 6 hours, microbes treated with the Example 7 barrier-forming composition failed to regrow during the same time period post-exposure. Similarly, exposure of Candida cells to the Example 7 barrier-forming composition completely inhibited growth during the incubation period (data not shown).

These results further confirmed that the barrier-forming composition possesses prolonged antimicrobial activity.

### Examples 164-166

In Vivo Study: Barrier-Forming Composition (Example 7) Lowers the Oral Microbial Load in Humans: Short- and Long-Term Activity

Short-Term Activity

The duration of activity of Example 7 was determined in healthy individuals by evaluating the effect of a single application on microbial burden of the oral cavity. In Examples 164-166, three healthy individuals (over 18 years of age, healthy mouth) were enrolled with informed consent, and asked to apply a single application of the composition of Example 7 on their cheeks. A single application was defined as three sprays of 0.25 ml each in volume. Next, swabs were collected from these individuals at baseline (pre-treatment), 1 hour, 2 hours, and 6 hours post-treatment. Swabs were cultured on agar media plates specific for aerobic or anaerobic organisms, incubated for 24-28 hours at 37° C., and the number of CFUs were counted. Effect of Example 7 on microbial burden was determined (CFUs), and percentage inhibition was calculated for each post-exposure time point relative to the baseline (0 minutes) CFUs.

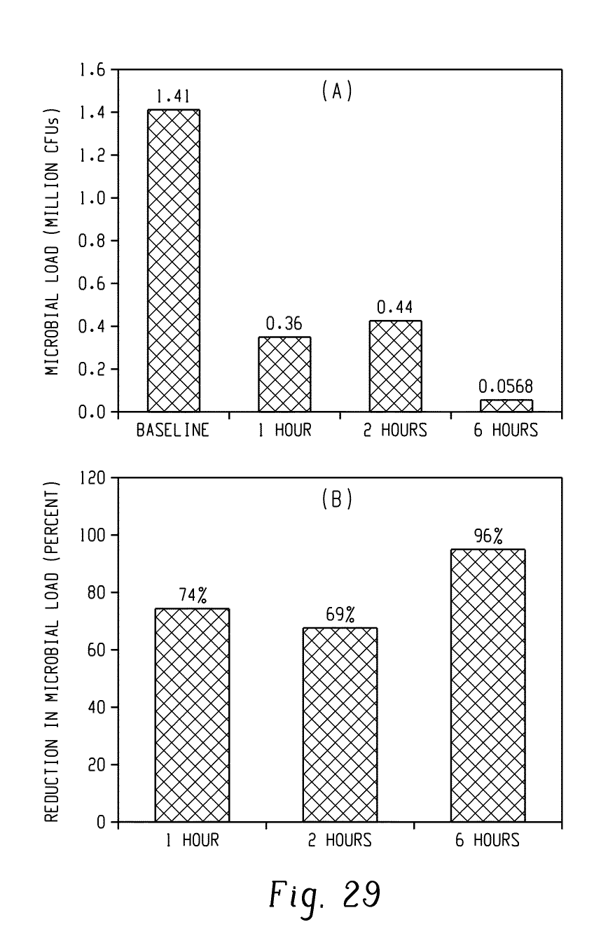

The results showed that application of Example 7 led to consistent reduction in microbial load for up to 6 hours (See FIG. 29A, which shows CFUs of a representative tested individual. Treatment with the barrier-forming composition resulted in 69% to 96% reduction of the microbial burden in the oral cavity (See FIG. 29B, which shows a representative individual's reduction in microbial load.)

### Examples 167-169

The activity of the barrier-forming composition over a 5-day period against oral microbes was evaluated. In Examples 167-169, three healthy individuals were enrolled, and asked to apply a single dosage (three sprays 0.75 mLs total) of Example 7 three times daily (approximately 9 AM, noon, and 3 PM) for a 5-day period (representing a typical 5-day work-week). Swabs were collected from these individuals at baseline (before application on day 1) and at the end of the day on each day during the 5-day period. Collected swabs were cultured on agar media plates, incubated for 24-28 hours at 37° C. and at 5% CO₂ humidity, and the number of CFUs were counted.

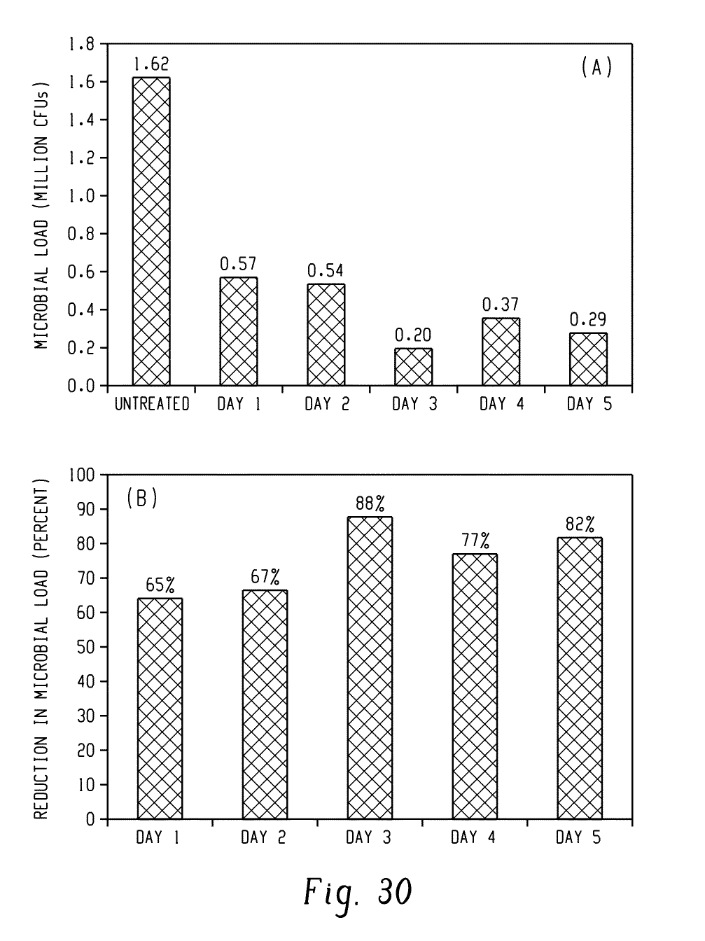

The effect of the Example 7 barrier-forming composition on microbial burden was determined (as median CFUs for the three subjects), and percentage inhibition was calculated for each post-exposure time point relative to the baseline (0 min) CFUs. FIG. 30 shows these results in a graph of CFUs versus time (FIG. 30A) and reduction in microbial load versus time (FIG. 30B). Examples 167-169 demonstrate that application of Example 7 over 5 days led to consistent reduction in microbial load over the 5-day test period (FIG. 30A). Treatment with the Example 7 barrier-forming composition resulted in 65%-88% reduction of the median microbial burden in the oral cavity of the study participants (FIG. 30B).

### Examples 170-198

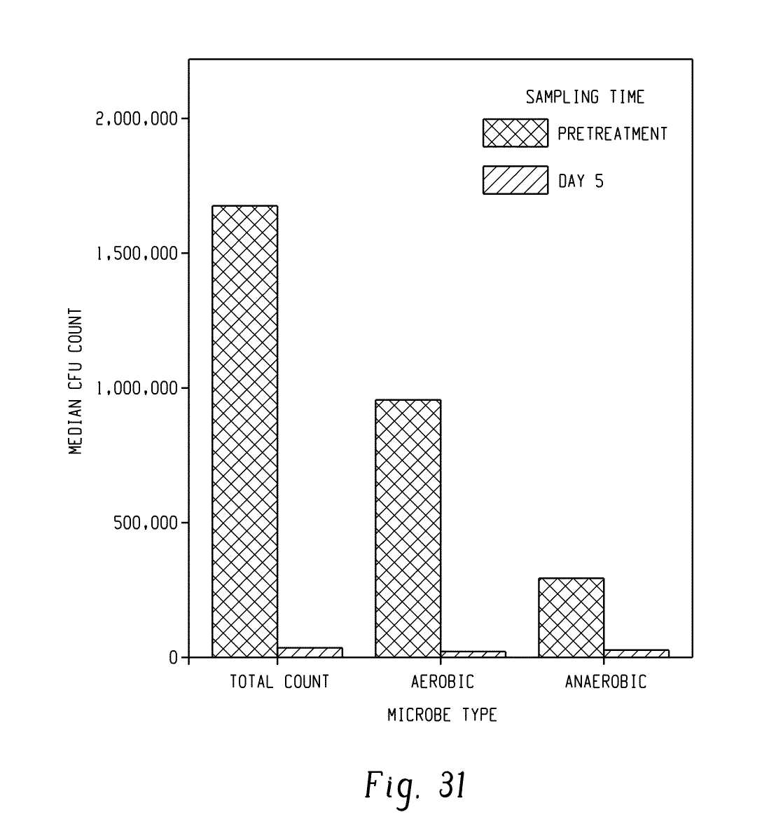

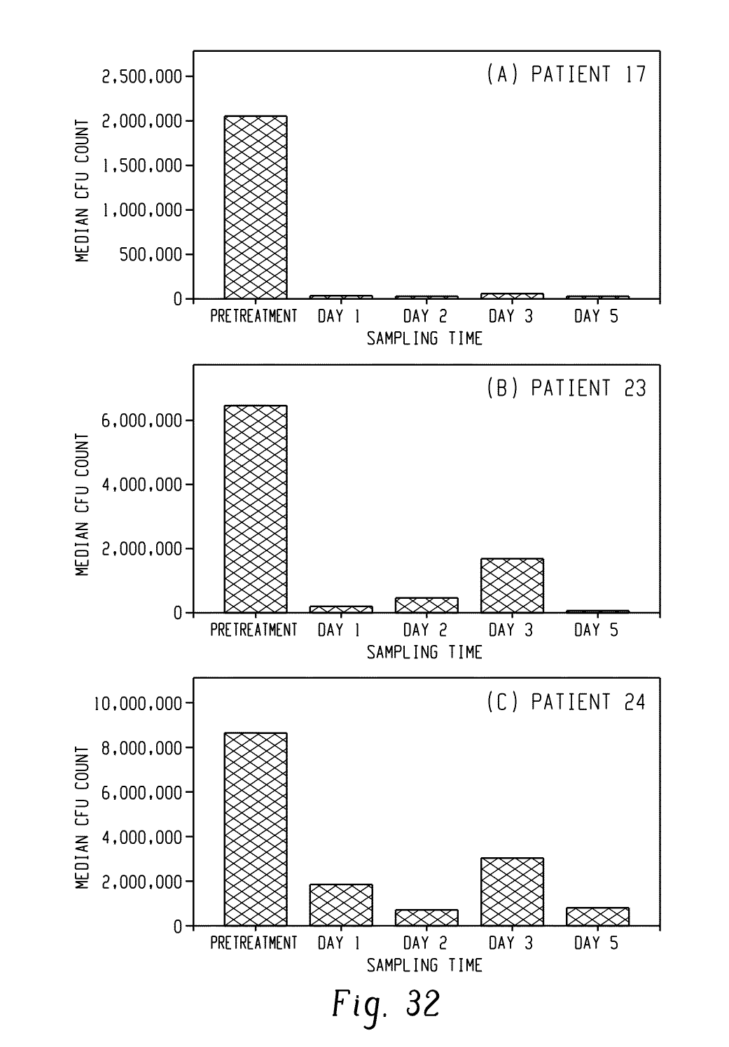

In a clinical study, twenty-nine healthy individuals were enrolled after informed consent. Baseline information was recorded (age in years, gender, ethnicity, and date of enrolment). Oral examination of the mouth was undertaken, and the inside of the mouth (cheek) was swabbed with a sterile culture swab. Baseline oral swab samples were cultured to determine bacterial load prior to study. In Examples 170-198, each of the twenty-nine participants were given a spray bottle containing the barrier-forming composition of Example 7 and instructed to spray the inside of their mouth for a total volume of 0.75 ml, then swish for 30 seconds and swallow. Two groups of approximately equal number of participants were tested. One group used the example barrier-forming composition every two hours, three times a day, for five days (a typical work week). The other group used the example barrier-forming composition every two hours, four times a day, for five days (a typical work week). No substantial difference was noted in the two groups. Swabs were collected on days 1, 2, 3, and 5 at the end of the day (8 hours after the first administration of the barrier-forming composition) and cultured on media specific for aerobic and anaerobic bacteria. Data were presented as number of microbes: total, aerobic and anaerobic. FIG. 31 shows a graph of total microbial load and breaks down the total into aerobic and anaerobic counts from just prior to treatment and on day 5 of treatment. FIG. 32 shows graphs of microbial load over the 5 day period in oral samples obtained from three representative study participants.

Overall, the in vivo testing showed that the barrier-forming composition exhibits antimicrobial activity against oral microbes, as measured by reduction in the levels of these organisms, over both short- and long-term duration.

The data showed that treatment with the barrier-forming composition over a 5-day period resulted in reduction in the oral microbial load, for total microbes, aerobic and anaerobic organisms.

### Example 199-205

Identification of Additional Humectants for Forming a Barrier to Prevent Microbial Penetration

In Example 199 an in vitro filter insert-based model (see FIG. 33) was used to test different humectants at different concentrations.

Six compositions were prepared according to Table XII based on the mixing procedures used for Examples 3-8.

TABLE XII

| | Ex. | Ex. | Ex. | Ex. | Ex. | Ex. | Ex. |
|---|---|---|---|---|---|---|---|
| | 199 | 200 | 201 | 202 | 203 | 204 | 205 |
| Xanthan | 0.4 | 0.4 | 0.4 | 0.4 | 0.4 | 0.4 | |
| Gum | | | | | | | |
| Glycerin | 4.5 | 4.5 | 4.5 | | | | 4.5 |
| Sorbitol | | 4.5 | 4.5 | 4.5 | | 4.5 | |
| Xylitol | | | 4.5 | 4.5 | 4.5 | | 4.5 |

Next, 100 μL of Examples 199-205 were placed into filter inserts (pore size 0.8 μm diameter, that allows both bacteria and fungi to pass through) and allowed to form a layer. Next, organisms were overlaid on the layer formed by the test solutions. The filter inserts containing the layer of test solutions and microorganisms were then placed on the surface of agar media plates and incubated for 24 hours at 37° C. After the incubation period, the agar media plates were evaluated for growth on filter insert and in the agar media. Growth on filter insert but no growth in agar media indicated that the test solution formed a barrier, which prevented the microbes from passing through. In contrast, microbial growth in the filter insert as well as the agar media indicated that no such barrier was formed.

The results showed that each of the xanthan gum-based solutions containing the tested humectants (singly or in combination) formed intact barriers on the filter insert that prevented the passage of microorganisms into underlying agar medium.

### Example 206-213

Determination of the Solubility Limits of Xanthan Gum

To determine the solubility of xanthan gum, it was mixed at different concentrations in water and the solubility observed by monitoring the presence or absence of clumps and free flow of the mixture. Table XIII reports the results and concentrations.

TABLE XIII

| | Xanthan Gum | |
|---|---|---|
| Example | Concentration | Solubility |
| 206 | 0.40% | free flowing viscous solution |
| 207 | 0.45% | some clumps, viscous solution |
| 208 | 0.5% | more clumps, viscous solution |
| 209 | 0.6% | clumps, more viscous than above |
| 210 | 0.7% | clumps, more viscous than above |
| 211 | 0.8% | Extensive clumps, highly viscous solution, |
| | | no free flow |
| 212 | 0.9% | Extensive clumps, highly viscous solution, |
| | | no free flow |
| 213 | 1.00% | Extensive clumps, highly viscous jelly, |
| | | no free flow |

We found that when mixed at 0.4%, xanthan gum formed a free-flowing solution (Table XIII). In contrast, mixtures containing 0.45% or 0.5% xanthan gum formed a viscous fluid but contained small clumps. The extent of clumps increased with increasing concentration of xanthan gum (0.6% and 0.7%). At concentrations>0.8%, xanthan gum mixture contained extensive clumps, with a jelly-like consistency and no free flow.

### Example 214

Comparison of Cationic CPC in Barrier-Forming Composition with Neutral Antimicrobial Agent in Barrier-Forming Composition

In Example 214, the formulation of Example 7 was made, except the neutral agent Citral was used instead of CPC. The antimicrobial activity of formulations containing CPC (0.1%) or Citral (0.5%) against Streptococcus was ascertained. The assay described above in Examples 48-61 was used to perform these studies.

The results showed that the formulation containing citral exhibited antimicrobial activity (MIC=12.5%). However, activity of formulation containing citral was significantly less potent than that containing CPC (MIC=0.098%).

### Example 215

Physico-Chemical Testing of Hydrophobicity and Comparison

In Example 215 thin layer chromatography analysis was used to compare the hydrophobicity of Example 7 with a hydrophobic composition. The hydrophobic composition was comprised of the components in Table XIV.

TABLE XIV

| | Wt % |
|---|---|
| Glycerin | 7 |
| Sorbitol | 5 |
| Poloxamer 338 | 1 |
| PEG 60 Hydrogenated castor oil | 1 |
| VP/VA copolymer | 0.75 |
| Sodium benzoate | 0.5 |
| Cellulose Gum | 0.2 |
| CPC | 0.05 |
| Methyl Paraben | 0.05 |
| Propyl paraben | 0.05 |
| Sodium Saccharin | 0.05 |
| Xanthan Gum | 0.01 |
| Disodium Phosphate | 0.006 |
| Flavoring and coloring agents | 0.121 |

*the remainder of the composition was purified water

10 μL of Example 7 and the hydrophobic composition were deposited on pre-made TLC plates (at a distance of 2 cm from the bottom edge). The spots were air-dried for 5 minutes, and the plates were placed in a TLC chromatography jar containing water as a solvent. The TLC system was allowed to run until the solvent front reached the top edge of the plate. Plates were removed and the solvent and sample fronts were marked. The Relative Front (Rf) values were calculated for the two samples using the formula II:

Rf=Distance traveled by spot/Distance traveled by solvent front II

The results showed that the Rf value for the hydrophobic composition and Example 7 were 0.33 and 0, respectively, indicating that the hydrophobic composition was highly miscible in water. In contrast, Example 7 did not exhibit any mobility in the aqueous solvent, demonstrating that this formulation is hydrophobic or not hydrophilic.

### Example 216

A barrier-forming composition was made by mixing the components according to Table XV below in water to form a solution. A eucalyptol component was also included in an amount of 5× per the Homeopathic Pharmacopeia, but also did not affect the test results, other than demonstrating that the composition still works with this component added into it. All percentages are by weight.

TABLE XV

| | Antimicrobial | | Gum |
|---|---|---|---|
| | (CPC) | Humectant (Glycerin) | (Xanthan Gum) |
| Example 216 | 0.01% | 35% | 0.4% |

### Examples 217-219

The barrier-forming composition was also shown to have effectiveness in killing allergy causing molds. MIC tests were performed on a polystyrene plastic surface.

In Example 217 the barrier-forming composition of Example 216 was tested to determine its MIC against Stachybotrys MRL 9740. The Example 7 composition had an MIC of 0.06 micrograms/ml.

In Example 218 the barrier-forming composition of Example 216 was tested to determine its MIC against Aspergillus fumigatus 18748. The Example 7 composition had an MIC of 0.49 micrograms/ml.

In Example 219 the barrier-forming composition of Example 216 was tested to determine its MIC against Cladosporium. The Example 7 composition had an MIC of 0.39 micrograms/ml.

Because Stachybotrys and Aspergillus fumigatus are mold-causing organisms, these examples further support the embodiment wherein the barrier-forming composition is applied to surfaces to prevent or treat mold growth or discoloration.

### Examples 219-224

In Examples 219-224 the effect of barrier-forming composition on MRSA biofilm formation on a silicone elastomer disc surface was evaluated.

In Examples 219-221, three silicone elastomer discs with a 1 cm diameter were pre-sprayed with 0.25 mL with the Example 7 barrier-forming composition for 60 min and incubated at 37° C. In Examples 222-224a control example was performed by treating a silicone elastomer disc with an equivalent amount of a phosphate-buffered saline (PBS) for 60 minutes and incubated at 37° C.

The Example 219-224 pretreated discs were each submerged in 4 mL MRSA suspension (1×10⁷ cells/mL), and incubated at 37° C. for 90 min (“Adhesion Phase”). Next, the discs with adherent cells were removed and transferred to wells containing 4 mL of Brain Heart Infusion (BHI). The wells were incubated at 37° C. on a rocker for 24 hours. Biofilm formation on the discs was evaluated by quantitative culturing on BHI agar plates. Scanned images of the wells were recorded using a scanner.

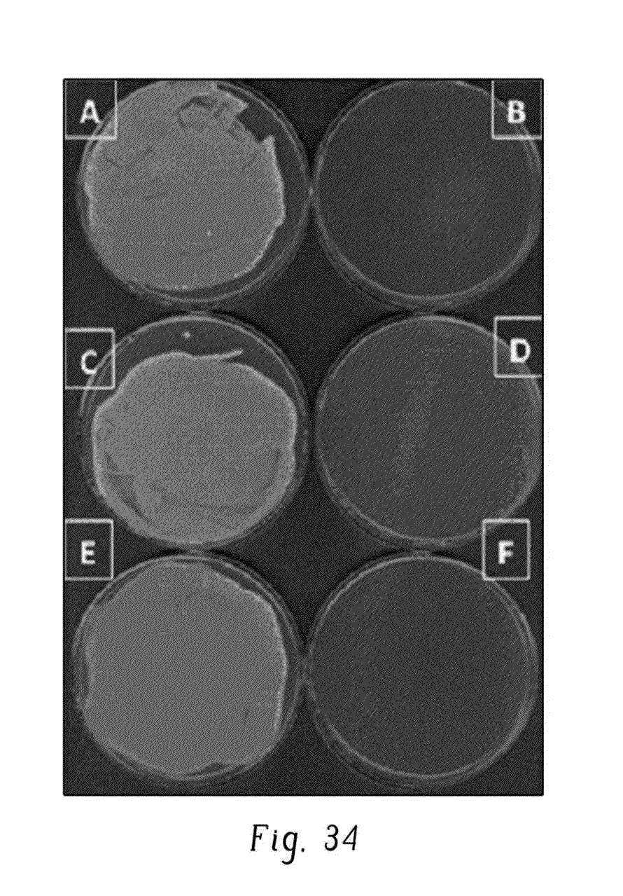

As shown in Table XVI, pre-treatment with Example 7 barrier-forming composition prevented formation of biofilms on the disc surface. FIG. 34 shows images of colony burden in biofilms formed by MRSA on the PBS treated (A, C, E) and Example 7 treated (B, D, F) discs.

TABLE XVI

| Treatment | Example | MRSA CFUs/mL |
|---|---|---|
| Example 7 | 219 | 0 |
| barrier- | 220 | 0 |
| forming | 221 | 0 |
| composition | | |
| PBS | 222 | 1.58 × 10⁸ |
| | 223 | 1.72 × 10⁸ |
| | 224 | 1.53 × 10⁸ |

### Examples 225-246

The Example 7 barrier-forming composition was tested to determine its efficacy against several strains of Bordetella pertussis. In test Examples 225-235, agar-based assays were constructed in which Example 7 barrier-forming compositoin was incorporated in Regan-Lowe Charcoal agar BBL #297883 plates as a 64 microgram/mL dilution in water. Control Examples 236-246 were agar plates containing no Example 7 barrier-forming composition. In each of Examples 225-246 5×10⁴ cells (50 uL) of Bordetella pertussis were spotted on the test surface and plates were incubated at 37 degrees C. for 24 hours. As shown in Table XVII, confluent growth was observed in control Examples 236 to 246, while no growth was observed in test Examples 236-246. The designation 4+ means luxurious growth.

TABLE XVII

| | Bordetella | Microbial |
|---|---|---|
| Example | pertussis Strain # | Growth |
| 225 | J11E | None |
| 226 | J11F | None |
| 227 | J14B | None |
| 228 | J14C | None |
| 229 | J14D | None |
| 230 | J14G | None |
| 231 | J32B | None |
| 232 | J32C | None |
| 233 | J32D | None |
| 234 | J36E | None |
| 235 | J36F | None |
| 236 | J11E | 4+ |
| 237 | J11F | 4+ |
| 238 | J14B | 4+ |
| 239 | J14C | 4+ |
| 240 | J14D | 4+ |
| 241 | J14G | 4+ |
| 242 | J32B | 4+ |
| 243 | J32C | 4+ |
| 244 | J32D | 4+ |
| 245 | J36E | 4+ |
| 246 | J36F | 4+ |

### Examples 247-252

The antiviral activity of the barrier-forming composition, Example 7 (in various diluted concentrations) was evaluated against the ATCC VR-1200 strain of rhinovirus.

Human Hepatoma (HUH-7) Cells were prepared in 24-well plates with Dulbecco's Modified Eagle Medium (DMEM) with 10% heat inactivated fetal calf serum and supplemented with L-glutamine (Lglu) and penicillin/streptomycin (P/S) (unless specified, all reagents produced by Gibco, N.Y., USA). All culture cells were grown to 90-100% confluence at 37° at 5% CO₂ and then washed with OptiMEM+P/S+Lglu once prior to infection.

In Examples 247-251, the Example 7 composition was applied to cell monolayers at varying concentrations (5%, 10%, 15%, 20%, 50% diluted in 400 microliter optiMEM (+P/S, +Lglu)) for a working CPC concentration of 0.005%, 0.01%, 0.015%, 0.02% and 0.05% respectively, and was allowed to dwell for 1 hour prior to inoculation. In control Example 252 400 microliter optiMEM (+P/S,+Lglu) was applied to the cells and allowed to dwell for 1 hour prior to inoculation.

The cell monolayers were then removed from the Example 7 dilutions or control optiMEM and rhinovirus was applied at a multiplicity of infection (MOI) of 0.1. Cells were incubated with virus at 32.5° C. for 1 hour. After which the inoculum was removed and 500u1 OptiMEM+P/S+Lglu was placed on the cells. Cells were then grown at 32.5° C. at 5% CO2. After 5 days incubation, cell culture supernatants were collected for rhinovirus viral load quantification.

Rhinovirus viral titer of the Example 247-251 cell culture supernatants were measured by real time PCR. In comparison to Control Example 252 significantly decreased rhinovirus viral load was demonstrated in Example 251, which was a 50% concentration of Example 7. See Table XVIII below.

TABLE XVIII

| Example | Wt. % Example 7 | Amount | Viral load/mL |
|---|---|---|---|
| 247 | 5% | 303354.64 | 12141854.69 |
| 248 | 10% | 5628.209 | 2251283.75 |
| 249 | 15% | 92717.83 | 37087131.25 |
| 250 | 20% | 8776.60 | 3510638.67 |
| 251 | 50% | 0 | 0 |
| 252 | 0 (control) | 95307.36 | 38122943.75 |

### Examples 253 and 254

A test Example 253 was formulated with a 50% Example 7 diluted suspension (0.05 CPC concentration) in 500 microliter optiMEM (+P/S,+Lglu). A control Example 254 was formulated as a control solution with no Example 7 (500 microliter optiMEM (+P/S,+Lglu)). Examples 253 and 254 were applied the cells disclosed in Examples 246-252, but at defined intervals: T-1 hour, T-30 min, and T-0 (Immediate) prior to infection.

The cell monolayers were then removed from the Example 253 suspension and the Example 254 control solution. The rhinovirus viral particles were applied to the treated cell monolayers at a multiplicity of infection (MOI) of 0.1. Cells were incubated with virus at 32.5° C. for 1 hour. After which the inoculum was removed and 500 ul OptiMEM+P/S+Lglu was placed on the cells. Cells were then grown at 32.5° C. at 5% CO₂ for 5 days. The cells treated with Example 253 and 254 were viewed daily for the presence of cytopathic effect. After 5 days incubation, cell culture supernatant was gathered for immunofluorescence and rhinovirus viral load quantification.

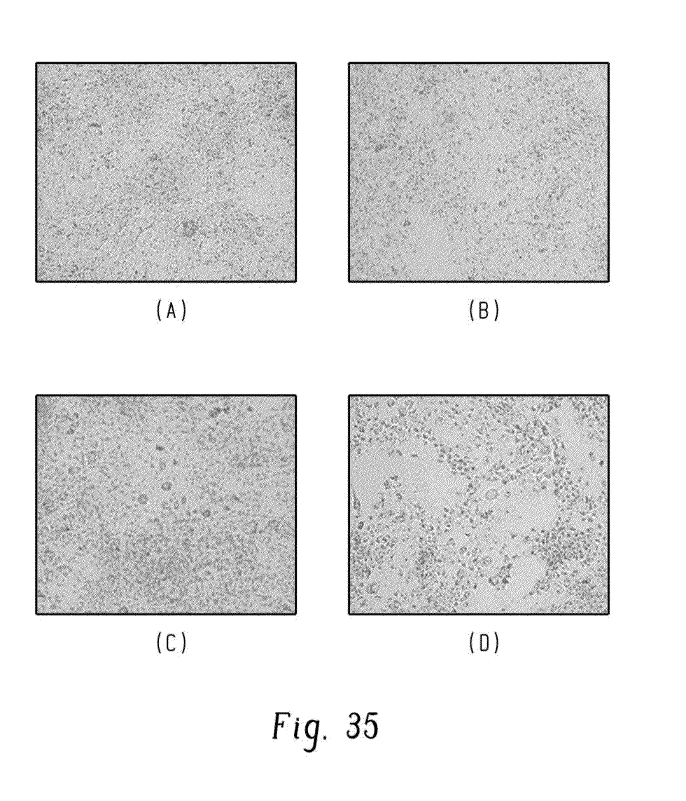

FIG. 35 discloses photos of cells treated with test Example 253 at FIG. 35(a) T-1 hr, FIG. 35(c) T-30 min and FIG. 35(b) T 0 (immediate). None of these photos demonstrated any cytopathic effect and healthy cells overgrew the plate. However, as shown in FIG. 35(d) the Example 254 untreated control cells demonstrated focal rounding, detachment and cell death. Cytopathic effect determination included the development of focal rounding, cell size enlargement or reduction, syncytial formation, development of multinucleated giant cells, and detachment.

Immunofluorescence was determined as follows: Virus infected cell monolayers and uninfected control were washed with sterile PBS. The cells were trypsinized, spotted upon wells on slides and fixed with acetone. The slides were tested by DFA employing FITC labeled monoclonal antibodies. An indirect immunofluorescence assay was performed using Light Diagnostics Pan-Enterovirus Detection Kit (Millipore). This detection kit is well described for having cross reactivity with rhinovirus infected cells. All antibody dwell steps occurred for 1 hour at 37° C. Following a final wash, cells were evaluated at a wavelength of 488 nm for the presence of fluorescence.

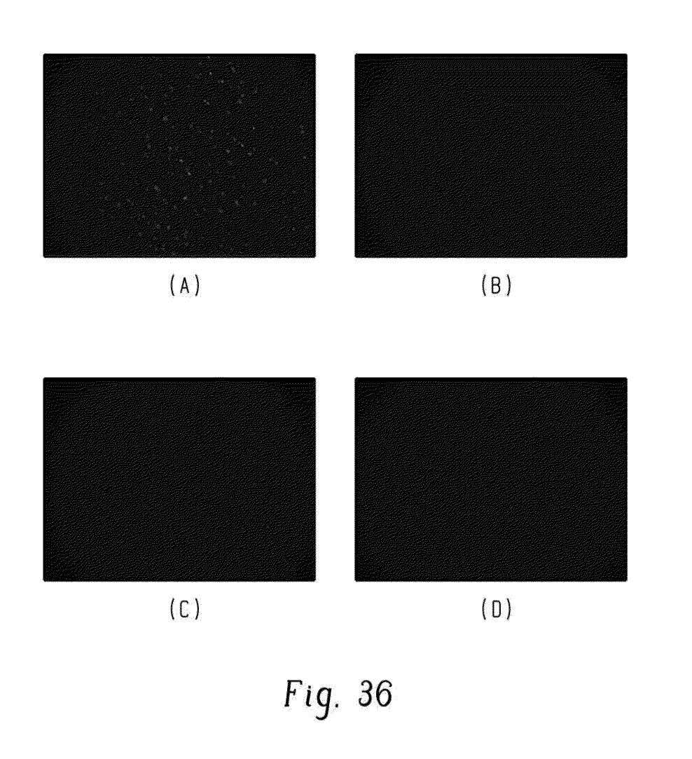

FIG. 36 discloses immunofluorescence photos of cells pretreated treated with test Example 253 at FIG. 36(d) T-1 hr, FIG. 36(b) T-30 min and FIG. 36(c) T-0 (immediate). The cells treated with Example 253 for 1 hour and 30 minutes displayed no immunofluorescence. The cells treated with Example 253 for T-0 (immediate) demonstrated scant fluorescence. However, the untreated control Example 254 showed substantial immunofluorescence suggesting profound viral infection (FIG. 36(a)).

Viral load for the samples was quantified as follows: Cell culture supernatants were collected and stored at −80° C. Nucleic acid was extracted using QIAamp Viral RNA Kit (QIAGEN, Valencia, Calif.). Random hexamer primers (Invitrogen Carlsbad, Calif.) were used to create a cDNA library for each specimen. Reverse transcription reactions were performed with M-MLV RT (Invitrogen, Carlsbad, Calif.) according to the manufacturer's specifications. Quantitative analysis was performed on a StepOne Plus Taqman Real Time PCR (Applied Biosystems, Branchburg, N.J.) using TaqMan Universal PCR Master Mix (Applied Biosystems, Branchburg, N.J.), 2 microliter of cDNA sample, and primers/probes targeting the rhinovirus polyprotein gene. A reference standard was prepared using an amplicon amplified by conventional RT-PCR, gel purified (QIAquick, Qiagen, Valencia, Calif.), and quantified using a spectrophotometer (Beckman Coulter, Brea, Calif.). The results are shown in Table XIX.

TABLE XIX

| | Amount | Viral load/mL |
|---|---|---|
| Example 253: | 0 | 0 |
| 1 hour pretreatment | | |
| Example 253: | 0 | 0 |
| 30 minute pretreatment | | |
| Example 253: | 0 | 0 |
| Immediate pretreatment | | |
| Example 254 (control) | 331025.2 | 1.32 × 10⁸ |

No rhinovirus amplification was apparent at T-1 hour, T-30 min, or T-0 (immediate) timepoints at 5 day post infection. Untreated (control) cells demonstrated substantial amplification (>10⁸ copies/ml) suggesting viral infection.

It is claimed:

1. A method for reducing risk of infection from microorganisms for certain elevated-risk individuals, the method comprising:
administering a barrier-forming composition in a therapeutically effective amount to a surface, the surface comprising a mucosa, or a mucosal or skin lesion of an individual;
the individual having an elevated risk condition: the elevated risk condition being selected from the group consisting of: an elevated risk of being exposed to harmful viral, bacterial, or fungal microorganisms; a condition causing an elevated risk of infection from viral, bacterial, or fungal microorganisms; and an elevated risk of serious complications resulting from an infection caused by viral, bacterial, or fungal microorganisms;
forming a barrier coating on the surface that is active to trap, and kill or neutralize microorganisms encountered by the barrier coating and effectively reducing the microbial load on the surface;
continuing to administer the barrier-forming composition in a therapeutically effective amount to the surface, for one time or more in a 24 hour period.

2. The method of claim 1, wherein the barrier coating is active to trap, and kill or neutralize microorganisms encountered by the barrier coating for a duration of at least one hour.

3. The method of claim 1, wherein the individual has the elevated risk of being exposed to harmful viral, bacterial, or fungal microorganisms due to the individual living or working on an airplane, train, bus, ship, boat, in a school, a library, a dormitory, a hotel, an apartment building, a courthouse, a correctional facility, an airport, a restaurant, a movie theater, a theater, a mall, a retail establishment, a special event arena, a special event stadium, a religious gathering place, a nursing home, hospital, other health-care facility, an office, or day-care facility.

4. The method of claim 1, wherein the individual has the condition causing an elevated risk of infection from viral, bacterial, or fungal microorganisms; the condition being one or more selected from the group consisting of: having received an organ or bone marrow transplant, having received a skin graft, having undergone surgery within 30 days, undergoing ventilator treatments, HIV and AIDS, cancer, auto-immune disease, diabetes, conditions that cause lesions on the skin or mucosa, and having lesions on the skin or mucosa.

5. The method of claim 1, wherein the individual has the elevated risk of serious complications resulting from an infection caused by viral, bacterial, or fungal microorganisms, the elevated risk being due to at least one condition selected from the group consisting of: being immunocompromised, having received an organ or bone marrow transplant, having received a skin graft, undergoing chronic steroid treatment, being under 19 and undergoing long-term aspirin treatment, being morbidly obese, being pregnant, being 65 years of age or older, children younger than two years of age, HIV, AIDS, cancer, metabolic disorder, mitochondrial disorder, liver disorder, kidney disorder, asthma, blood disorders, endocrine disorders, heart disease, chronic lung disease, cerebral palsy, epilepsy, stroke, muscular dystrophy, and spinal cord injury.

6. The method of claim 1, further comprising, administering a therapeutically effective amount of the barrier-forming composition every about two to about twelve hours to the surface.

7. The method of claim 1, further comprising, administering a therapeutically effective amount of the barrier-forming composition one more times in a 24 hour period for five 24 hour periods or more.

8. The method of claim 1, further comprising, administering a therapeutically effective amount of the barrier-forming composition three or more times in a 24 hour period for five 24 hour periods or more.

9. The method of claim 1, wherein the barrier-forming composition comprises a humectant, an antimicrobial, and optionally a gum, wherein the gum, if present, is present in an amount of about 0.01% to about 0.4% by weight of the total barrier-forming composition.

10. The method of claim 1, wherein the barrier-forming composition meets the following requirements:
about 0.0001%≦C≦about 0.4%;
about 0.07%≦H≦about 70%; and
0.0005%<A or
about 0%≦C≦about 0.4%;
about 55%≦H≦about 70%; and
0.0005%<A
wherein all percentages are by weight of the total composition;
wherein C is a carbohydrate gum; H is a humectant; and A is the antimicrobial agent.

11. The method of claim 1, wherein the antimicrobial agent is a monoquaternary ammonium compound.

12. The method of claim 1, further comprising:
wherein the step of administering the barrier-forming composition occurs in response to:
a. identifying a contaminated environment that the individual with the elevated risk condition is present in or is going to be present in, wherein the contaminated environment is known or expected to be contaminated with harmful viral, fungal, or bacterial microorganisms; or
b. observing a contamination event in an environment wherein the individual with the elevated risk condition is present in the environment or is going to be present in the environment;

13. The method of claim 1, wherein the barrier-forming composition has a viscosity of less than 500 cps.

14. The method of claim 1, wherein the therapeutically effective amount of the barrier-forming composition is non-toxic for human consumption.

15. The method of claim 1, wherein the barrier layer has antimicrobial cidal or static activity for a duration of about six to about twenty-four hours.

16. The method of claim 1, wherein the barrier-forming composition is applied by spraying.

17. The method of claim 1, wherein the barrier-forming composition is applied by a roll-on applicator or a treated wipe.

18. The method of claim 1, further comprising administering the barrier-forming composition every day at least three times a day.

19. The method of claim 1, wherein the microorganisms are selected from the group consisting of: Candida, Pneumonia, MRSA, Streptococcus, P. gingivalis, S. pyogenes, S. pneumonia, S. mutans, S. aureus, Y. enterocolitica, Acinetobacter bumanii, Streptococcus sanguis, S. oralis, S. mitis, S. salivarius, S. gordonii, Aggregatibacter actinomycetemcomitans, Fusobacterium nucleatum, C. albicans, C. krusei, C. tropicalis, C. glabrata, microorganisms that cause upper respiratory infections, HIV, EBV, and influenza viruses.

20. The method of claim 1, wherein the microorganisms are selected from the group consisting of: Candida, Pneumonia, MRSA, Streptococcus, P. gingivalis, S. pyogenes, S. pneumonia, S. mutans, S. aureus, Y. enterocolitica, Acinetobacter bumanii, Streptococcus sanguis, S. oralis, S. mitis, S. salivarius, S. gordonii, Aggregatibacter actinomycetemcomitans, Fusobacterium nucleatum, C. albicans, C. krusei, C. tropicalis, C. glabrata, HIV, EBV, influenza viruses, microorganisms that cause upper respiratory infections, Centipeda periodontii, Eikenella corrodens, Enterobacteriaceae, Fusobacterium nucleatum subsp. nucleatum, Fusobacterium nucleatum subsp. polymorphum, Fusobacterium nucleatum subsp. vincentii, Fusobacterium periodonticum, Porphyromonas endodontalis, Prevotella (Bacteroides) melaminogenica, Prevotella intermedia, Bacteroides (Bacteroides) loescheii, Solobacterium moorei, Tannerella forsythia (Bacteroides forsythus), Treponema denticola, Clostridium difficile, Bordetella pertussis, Burkholderia, Aspergillus fumigatus, Penicillium spp, Cladosporium, HPV, cold, Klebsiella pneumoniae, Salmonella choleraesuis, Escherichia coli (0157:H7), Trichophyton mentagrophytes, Rhinovirus Type 39, Respiratory Syncytial Virus, Poliovirus Type 1, Rotavirus Wa, Influenza A Virus, Herpes Simplex Virus Types 1 & 2, and Hepatitis A Virus.

21. The method of claim 1, wherein the antimicrobial agent is a monoquaternary ammonium compound.

22. The method of claim 1, wherein the antimicrobial agent is exclusive of fatty acid ethers or esters of polyhydric alcohols or alkoxylated derivatives thereof.

23. The method of claim 1, wherein there is about 50% to about 99% reduction of the microbial burden from about one to about six hours after the administering step.

24. The method of claim 1, wherein the mucosa is an oral, nasal, or pharyngeal mucosa.

25. A method for reducing the microbial load in immunocompromised individuals, the method comprising:
administering a barrier-forming composition that comprises an antimicrobial to a surface, the surface comprising an oral, nasal, or pharyngeal mucosa or a skin lesion of an immunocompromised individual;
forming a barrier coating on the surface that is active to trap, and kill or neutralize microorganisms encountered by the barrier coating for a duration of at least about one hour, thereby effectively reducing the microbial load of the surface;
wherein the barrier-forming composition meets the following requirements:
about 0.0001%≦C≦about 0.4%;
about 0.07%≦H≦about 70%; and
0.0005%<A or
about 0%≦C≦about 0.4%;
about 55%≦H≦about 70%; and
0.0005%<A
wherein all percentages are by weight of the total composition;
wherein C is the carbohydrate gum; H is the humectant; and A is the antimicrobial agent.

26. The method of claim 25, wherein the barrier-forming composition has antimicrobial cidal or static activity for a duration of about six to about twenty-four hours.

27. The method of claim 25, wherein the barrier-forming composition has a viscosity of less than 500 cps.

28. The method of claim 25, further comprising continuing to administer the barrier-forming composition in a therapeutically effective amount to the surface, for one time or more in a 24 hour period.

29. The method of claim 25, wherein there is about 50% to about 99% reduction of the microbial burden from about one to about six hours after the administering step.
